(19) United States
(12) Patent Application Publication
(10) Pub. No.: US 2016/0024563 A1
(43) Pub. Date: Jan. 28, 2016

(54) METHOD FOR PERFORMING A MELTING CURVE ANALYSIS

(71) Applicant: QIAGEN GMBH, Hilden (DE)

(72) Inventors: Andy Wende, Hilden (DE); Ralf Himmelreich, Langenfeld (DE)

(21) Appl. No.: 14/782,496

(22) PCT Filed: Apr. 4, 2014
(86) PCT No.: PCT/EP2014/056765

§ 371 Date: Oct. 5, 2015

(30) Foreign Application Priority Data
Apr. 5, 2013 (EP) 13162534.5

(51) Int. Cl. C12Q 1/68

(52) U.S. Cl. CPC C12Q 1/6818; C12Q 1/6823

(57) ABSTRACT

The present invention related to methods for improving probe-based melting curve analysis methods. The improvement comprises the inclusion of a compound (A) which is a water-soluble polyanionic co-polymer comprising maleic acid, preferably poly(acrylic acid-co-maleic acid) (PAMA) in the analytical sample that is subjected to the melting curve analysis.

### FIELD OF THE INVENTION

The present invention pertains to methods, compositions and kits suitable for detecting and analysing target nucleic acids. In particular, the present invention pertains to improved methods for performing a probe-based melting curve analysis.

### BACKGROUND OF THE INVENTION

The detection, identification and analysis of nucleic acids comprised in a sample is an important field and many methods are available in the prior art for that purpose. In particular, amplification based analytical methods are widely used. The double-stranded amplicons obtained in an amplification reaction may be analysed by a melting curve analysis, wherein the dissociation characteristics of a double-stranded duplex are assessed during a gradual heating process. In a melting-curve analysis, a double-stranded duplex is gradually denatured (“melted”) to two single-stranded molecules by increasing the temperature in small increments and continuously measuring the dissociation of the double-stranded duplex into single strands. Thereby, a melting profile (also referred to as melting curve) characteristic for the double-stranded duplex is produced. The temperature at which DNA strands melt (separate) when heated can vary greatly, depending on the sequence, number of mismatches, length of the duplex and GC content. Even single-base differences in heterozygous DNA can change the melting profile. Thus, melting profiles can be used to identify and genotype DNA products. Usually, fluorescence based melting curve analysis methods are used. E.g. a melting curve analysis with a fluorescence-based readout can be performed in a real time PCR cycler. The obtained melting profile can be represented by plotting fluorescence (F) over temperature (T), or, to make the analysis more convenient, can be represented by the negative first derivative (−dF/dT versus T). The melting temperature Tₘ of a double-stranded duplex is defined as the temperature at which 50% of the molecules are double-stranded and 50% are single-stranded. The melting temperature Tₘ can be derived e.g. from the inflection point of the fluorescence (F) versus temperature (T) curves, or the peak value of the −dF/dT versus T curve. Tₘ is typically higher for double-stranded duplexes that are longer and/or have a high GC content while mismatches in the double-stranded duplex reduce the melting temperature and induce a shift in the obtained melting curve. Furthermore, Tₘ is influenced by the solution containing the double-stranded duplex, e.g. its ionic strength. A double-stranded duplex has in a defined setting a characteristic melting profile and melting temperature. Therefore, a melting curve analysis is often performed downstream of an amplification reaction in order to identify and/or verify the presence or nature of a target nucleic acid in a sample. E.g. the melting temperature or melting profile of an amplicon that was obtained in an amplification reaction can be determined in a melting curve analysis in order to verify that the obtained amplicon indeed corresponds to the expected amplification product. Melting curve analysis based methods are widely used in the research, medicine and diagnostic field e.g. in order to detect the presence or absence of a pathogen in a sample, to detect and/or categorize genetic mutations, in particular single nucleotide polymorphisms (SNPs), SNP genotyping, tumor typing, to identify new genetic variants without sequencing (gene scanning), to determine the genetic variation in a population (for example viral diversity) prior to sequencing, mutation discovery (gene scanning), heterozygosity screening, DNA finger printing, haplotype blocks characterization, DNA methylation analysis, DNA mapping, species identification, viral/bacterial population diversity investigation and HLA compatibility typing. Melting curve analysis based methods can be, depending on the used format, sensitive enough to allow the detection of a single base change difference between otherwise identical nucleotide sequences.

The melting temperature Tₘ is a convenient metric but is only one point on the melting curve. More information is contained in the complete melting curve (melting profile) than in the Tₘ. The shape of the melting curve is used extensively e.g. in sequencing matching and mutation scanning e.g. as an indicator of heteroduplexes formed from heterozygous DNA. The more profound the melting signal in form of a clear and narrow curve, the more meaningful is the assay. Therefore, it is important to obtain clear, clean curves in the melting curve analysis.

Different formats exist for performing a melting curve analysis. According to one format, melting curve analysis takes place by means of dyes that are specific for double-stranded DNA, for example by means of intercalating fluorescent dyes. During heating and thus denaturation of the double-stranded duplex, the fluorescent dye is released as the strands dissociate, and a decrease in fluorescence is recorded, thereby providing the melting profile. Examples of fluorescent dyes intercalating into double-stranded DNA are SYBR® Green and EvaGreen®. This format is e.g. used in forms of high resolution melting curve analysis, wherein the temperature increments are very small (e.g. 0.5° C. or less). However, this format has clear disadvantages because the dye binds to all double-stranded DNA duplexes present in the analytical sample that is subjected to the melting curve analysis. Therefore, the obtained melting profile includes all double-stranded amplicons comprised in the sample (including nonspecific amplicons or other non-target double-stranded molecules). Therefore, this format does not allow e.g. to focus the analysis on a specific double-stranded target amplicon or a small target region within a double-stranded amplicon. Furthermore, no multiplex analyses of different target amplicons or target regions within an amplicon can be performed with this format.

According to another format, a probe-based melting curve analysis is performed. Here, one or more probes are used that are capable of hybridizing to the target strand of the target amplicon. Upon hybridization to the target strand, the probes form a double-stranded duplex with the target strand of the amplicon, herein also referred to as target duplex. The target duplex formed between the one or more probes and the target strand is then analysed in a melting curve analysis. The use of probes is advantageous because it allows focusing the analysis of the target strand on the region that is covered by the probe, herein also referred to as target region. E.g. the target region can be a specific region of a target strand that is suspected to have nucleotide variation(s). This increases the sensitivity and makes probe-based melting curve analyses e.g. particularly suitable for the analysis of mutations, in particular single-nucleotide polymorphisms (SNP). This format can distinguish, e.g., between homozygous wildtype, heterozygous and homozygous mutant alleles by virtue of the melting profiles produced. Probes can be labeled or unlabeled. Target strand specific probes can be used in combination with double-stranded nucleic acid-specific dyes (see above). This is e.g. suitable if unlabeled probes are used. In addition to the full-length double-stranded amplicon that was formed during amplification, the double-stranded duplex formed between the probe and the target strand produces additional melting data that is, however, focused on the region under the probe (see e.g. Reed et al “High-resolution DNA melting analysis for simple and efficient molecular diagnostics” Pharmacogenomics (2007) 8(6), 597-608).

In a different, widely used format, labeled probes are used for performing the melting curve analysis. Examples of such labeling are fluorescent labels or quantum dots. In a widely used format, probes comprising a quencher and a fluorophore (e.g. molecular beacons or TaqMan® probes) are used for melting curve analysis. When the probes are hybridized to the target strand, fluorescence is emitted. As the probe detaches from the target strand during denaturation, the fluorescence decreases, thereby allowing to determine the melting profile by recording the decrease in fluorescence. When appropriately labeled probes are used, no double-stranded nucleic acid-specific dye is required as the signal necessary for obtaining the melting profile is provided by the labeled probes. This advantageously reduces the background and focuses the melting curve analysis on the target region(s) covered by the probe(s) and thus on the double-stranded target duplex. The double-stranded target amplicon or other double-stranded molecules which do not comprise a labeled probe do not emit fluorescence. A typical melting curve analysis method that is based on the use of probes includes performing an amplification reaction (e.g. PCR) to provide the double-stranded amplicon. If the probe was not already present during amplification, the probe is added after completion of the amplification and the resulting analytical sample is heated to e.g. at least 90° C. to separate the strands of the double-stranded amplicon. Then, the reaction is cooled to e.g. 45° C. or less in order to allow hybridisation of the probe to the target strand of the amplicon, thereby forming the target duplex. After hybridization, the analytical sample is gradually heated e.g. to about 80° C. in increments of e.g. 1° C. or less in order to melt the target duplex. The temperature can be maintained at each temperature level for a certain holding time (e.g. 1 s to 15 sec). Melting of the formed double-stranded target duplex is monitored as described above, e.g. by measuring the intensity of fluorescence emission in response to excitation by the PCR cycler's light source. Measurement can be performed continuously or at defined temperatures/temperature steps.

Inherent to probe-based melting curve analysis methods is the problem that the probe(s) which hybridize(s) to the target strand of the amplicon compete(s) with the complementary strand of the amplicon for hybridization. When performing a melting curve analysis of amplicons produced in a symmetric amplification reaction (wherein the forward and reverse primers are used in approx. equimolar concentration, thereby rendering approx. the same amount of target strand and complementary strand), this usually has the effect that low signals are obtained in the melting profile and that the obtained curve does not have a clear, narrow shape as is, however, desired for an accurate analysis. Furthermore, the results also vary depending on the used amplification buffer and sometimes, no melting profile can be determined at all. However, as discussed above, the more profound the melting signal in form of a curve, the more meaningful is the assay. Thus, a strong and clear melting curve is desired and also required for most applications. Therefore, when performing a probe-based melting curve analysis of double-stranded amplicons that were generated in a symmetric amplification, large amounts of double-stranded amplicon are needed in order to enhance the melting signal. However, in many cases this is not sufficient to obtain reliably strong signals, in particular when analysing mutations and/or when using multiplex formats. To overcome this problem it was common in the prior art to generate the amplicon for analysis by an asymmetric amplification. In an asymmetric amplification, the primer amplifying the target strand is used in excess of the primer which amplifies the complementary strand, thereby generating excess copies of the target strand as single-stranded amplicon. The complementary detection probe(s) can easily hybridize to the single-stranded target strand thereby increasing the visibility of the probe-target strand duplex melting transition (see e.g. Szilvasi et al, Clinical Biochemistry 38 (2005) 727-730, Reed et al, Pharmacogenomics 2007, 597-608; Huang, PLoS ONE April 2011, Volume 6, Issue 4 “Multiplex fluorescence melting curve analysis for mutation detection with dual-labeled, self-quenched probes). As a consequence, melting curves obtained from the analysis of target amplicons produced in an asymmetric amplification are generally strong and well defined. However, asymmetric amplification reactions generally hold the disadvantage of a reduced yield and less sensitivity as compared to symmetric amplifications.

It is the object of the present invention to improve probe-based melting curve analysis methods. In particular, it is the object of the present invention to provide an improved probe-based melting curve analysis method that allows to reliably perform a melting curve analysis also of amplicons generated in a symmetric amplification.

### SUMMARY OF THE INVENTION

The present invention is based on the surprising finding that the addition of a specific chemical to the analytical sample that is analysed by probe-based melting curve analysis significantly improves the results. In particular, the inventors found that an analytical sample which comprises a water-soluble polyanionic co-polymer which comprises maleic acid monomers, such as poly(acrylic acid-co maleic acid) (PAMA), produces reliable and strong signals in a probe-based melting curve analysis, even if the target amplicon to be analysed was obtained in a symmetric amplification reaction. Therefore, the present invention overcomes the need to obtain the target amplicon by an asymmetric amplification. Furthermore, also the performance on target amplicons obtained in an asymmetric amplification is improved. The present invention provides reliable results on crude amplification reactions that were obtained under different conditions. Therefore, the present invention makes an important contribution to the art by providing improved reliable methods for performing a probe-based melting curve analysis.

In a first aspect, the present invention provides a method for performing a probe-based melting curve analysis comprising
(a) preparing an analytical sample which comprises
   (i) at least one double-stranded target amplicon comprising a target strand and a complementary strand;
   (ii) a compound (A), which is a water-soluble polyanionic co-polymer comprising maleic acid; and
   (iii) at least one detection probe or at least one detection probe set capable of hybridizing to the target strand of the target amplicon;
   and wherein the analytical sample optionally comprises additionally the target strand as single-stranded amplicon;
(b) providing in the analytical sample a double-stranded target duplex comprising the detection probe or detection probe set hybridized to the target strand;
(c) gradually heating the analytical sample and measuring the dissociation of the double-stranded target duplex during heating.

As is shown by the examples, including compound (A) into the analytical sample significantly improves or even enables the generation of melting profiles in a probe-based melting curve analysis.

In a second aspect, the present invention provides a method for amplifying and detecting a target nucleic acid, comprising amplifying a target sequence of the target nucleic acid thereby providing a double-stranded target amplicon and performing a probe-based melting curve analysis as defined in the first aspect of the present invention.

In a third aspect, a composition is provided which comprises at least one detection probe or at least one detection probe set and a compound (A) which is a water-soluble polyanionic co-polymer comprising maleic acid. Such composition can be advantageously used in the method according to the first and second aspect. The composition can be e.g. added to the product of the amplification reaction in order to prepare the analytical sample for melting curve analysis. This is in particular feasible if no detection probe or detection probe set was present during the amplification reaction.

According to a fourth aspect, the present invention provides a kit for performing a probe-based melting curve analysis, wherein the kit comprises at least one detection probe or at least one detection probe set and a compound (A) which is a water-soluble polyanionic co-polymer comprising maleic acid. Said kit can be advantageously used in order to prepare the analytical sample for the probe-based melting curve analysis. Thus, the kit may be used in a method according to the first or second aspect of the present invention. Optionally, the kit may comprise further reagents such as e.g. one or more reagents for performing an amplification reaction.

According to a fifth aspect, the present invention pertains to the use of a compound (A) which is a water-soluble polyanionic co-polymer comprising maleic acid for preparing an analytical sample for melting curve analysis.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE FIGURES

FIG. 1 shows the melting curve profiles of crude PCR products. A melting peak is obtained only for the asymmetric PCR product.

FIG. 2 shows a melting curve obtained from analysis of either symmetric or asymmetric amplification products supplemented with EDTA to chelate Mg²⁺ ions. EDTA treatment does not render unpurified symmetric PCR products competent for melting curve analysis.

FIG. 3 a) to d) depict the melting curve profiles obtained from the analysis of analytical samples obtained from either symmetric or asymmetric unpurified amplification products which were after amplification supplemented with water (a), polyacrylic acid (b), polyvinylpyrrolidone K15 10000 (c) or poly (acrylic acid-co-maleic acid) (PAMA) (d). With symmetric PCR products, only analytical samples which included PAMA provided robust melting peaks.

FIGS. 4 a) and b) depict the melting curve profiles obtained from the analysis of analytical samples obtained from either symmetric or asymmetric unpurified amplification products which were after amplification supplemented with water (a) or (acrylic acid-co-maleic acid) (PAMA), betaine and EDTA (b).

FIGS. 5 a) and f) show the melting profiles obtained from analytical samples comprising unpurified PCR products of a symmetric PCR performed in various PCR buffer systems from QIAGEN. QuantiFast Probe PCR Mastermix (FIG. 5a), QuantiFast Multiplex Mastermix (FIG. 5b), QuantiTect Virus Mastermix (FIG. 5c), QuantiTect Probe PCR Mastermix (FIG. 5d), QuantiTect Multiplex Mastermix (FIG. 5e) and HotStarTaq Mastermix (FIG. 5f). No meaningful melting profile was obtained with analytical samples comprising water or no additives. In contrast, analytical samples comprising PAMA, betaine and EDTA provided clear melting profiles in all PCR-buffers tested.

FIGS. 6 a) and b) show the melting profiles obtained from analysis of symmetric PCR products supplemented with 0-0.1% (w/v) PAMA (FIG. 6a) and 0.15-0.5% (w/v) PAMA.

FIG. 7 shows the melting curve profiles obtained from a symmetric PCR reactions supplemented with PAMA and varying amounts of betaine.

FIG. 8 shows the melting curve profiles obtained from a symmetric PCR reactions supplemented with PAMA and varying amounts of EDTA.

FIG. 9 shows a photograph of reaction tubes with freeze-dried PAMA solutions. Freeze-dried PAMA is visible as white pellet at the bottom of the tubes.

FIG. 10 shows the melting profiles obtained from the analysis of crude symmetric PCR reactions supplemented with PAMA, with PAMA provided either in solution or in freeze-dried form. No difference can be seen between the obtained melting profiles.

### DETAILED DESCRIPTION OF THIS INVENTION

The present invention provides improved methods for performing a melting curve analysis. The invention is inter alia based on the finding that including a water-soluble polyanionic co-polymer comprising maleic acid such as PAMA in the analytical sample significantly improves the obtained melting profile. Thereby, the present invention also improves methods and assays, in particular in the diagnostic field that are based on or involve a melting curve analysis.

In a first aspect, the present invention provides a method for performing a probe-based melting curve analysis comprising
(a) preparing an analytical sample which comprises
   (i) at least one double-stranded target amplicon comprising a target strand and a complementary strand;
   (ii) a compound (A), which is a water-soluble polyanionic co-polymer comprising maleic acid; and
   (iii) at least one detection probe or at least one detection probe set capable of hybridizing to the target strand of the target amplicon;
   and wherein the analytical sample optionally comprises additionally the target strand as single-stranded amplicon;
(b) providing in the analytical sample a double-stranded target duplex comprising the detection probe or detection probe set hybridized to the target strand;
(c) gradually heating the analytical sample and measuring the dissociation of the double-stranded target duplex during heating.

The individual steps of said method as well as suitable and preferred embodiments will now be explained in further detail.

### Step (a)

In step (a), the analytical sample to be analysed in the melting curve analysis is prepared. The analytical sample comprises a double-stranded amplicon which comprises a target strand and a complementary strand.

The term “amplicon” in particular refers to a piece of nucleic acid, in particular double-stranded DNA, that was obtained as product of an amplification reaction such as e.g. a polymerase chain reaction. Here, the term amplicon is often used interchangeably with other common laboratory terms such as amplification product or PCR product. The term “a” target amplicon comprises, respectively refers to multiple copies of the same amplicon.

Step (a) may comprise performing an amplification reaction to produce at least one double-stranded target amplicon. Various amplification methods may be used in the context of the present invention. Suitable amplification methods include but are not limited to rolling circle amplification (such as in Liu, et al., “Rolling circle DNA synthesis: Small circular oligonucleotides as efficient templates for DNA polymerases,” J. Am. Chem. Soc. 118:1587-1594 (1996).), isothermal amplification (such as in Walker, et al., “Strand displacement amplification—an isothermal, in vitro DNA amplification technique”, Nucleic Acids Res. 20(7): 1691-6 (1992)), ligase chain reaction (such as in Landegren, et al., “A Ligase-Mediated Gene Detection Technique,” Science 241:1077-1080, 1988, or, in Wiedmann, et al., “Ligase Chain Reaction (LCR)—Overview and Applications,” PCR Methods and Applications (Cold Spring Harbor Laboratory Press, Cold Spring Harbor Laboratory, N Y, 1994) pp. S51-S64.), polymerase chain reaction, reverse transcription polymerase chain reaction (RT-PCR), microchip PCR, reverse transcription amplification, quantitative real time polymerase chain reaction (qPCR), NASBA, LAMP (loop mediated isothermal amplification), RPA (recombinase polymerase amplification), HDA (helicase dependent amplification), NEAR (nicking enzyme amplification reaction), TMA (transcription mediated amplification) and NASBA (nucleic acid sequence based amplification), allele specific polymerase chain reaction, polymerase cycling assembly (PCA), asymmetric polymerase chain reaction, symmetric polymerase chain reaction, linear after the exponential polymerase chain reaction (LATE-PCR), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination of the foregoing. Respective nucleic acid amplification technologies are well-known to the skilled person and, thus, do not need further description here. Preferably, the double-stranded amplicon was obtained in a polymerase chain reaction. Also a multiplex polymerase chain reaction can be performed to provide two or more target amplicons. As such amplification methods are standard in the art, they do not need any detailed description herein. However, non-limiting embodiments are also described herein.

The double-stranded amplicon may have been obtained by a symmetric or asymmetric amplification reaction. As explained in the background of the present invention, in a symmetric amplification reaction, the forward and reverse primers are used in an approx. equimolar concentration, thereby rendering approx. the same amount of target strand and complementary strand. Therefore, predominantly a double-stranded target amplicon is produced. In an asymmetric amplification, the primer amplifying the target strand is used in excess of the primer which amplifies the complementary strand, thereby generating excess copies of the target strand as single-stranded amplicon. When performing an asymmetric amplification reaction to provide the target amplicon, the analytical sample will accordingly comprise the target strand as single-stranded amplicon in addition to the double-stranded target amplicon. Therefore, according to one embodiment, the analytical sample comprises at least one double-stranded target amplicon comprising a target strand and a complementary strand and in addition thereto the target strand as single-stranded amplicon. Asymmetric amplification reactions such as asymmetric PCR reactions may be used for the generation of a surplus of the target strand. However, asymmetric PCR reactions generally hold the disadvantage of lower sensitivity and lower amplicon yield as compared to symmetric PCR reactions. Though preference is given to symmetric PCR reactions, the method of the present invention may also be used for the detection of asymmetric PCR products. Furthermore, as is demonstrated by the examples provided herein, it is an important advantage of the present invention that it is not necessary to perform an asymmetric amplification reaction in order to obtain strong, clear signals in a probe-based melting curve analysis. Due to the addition of compound (A), strong, reliable signals are also obtained if a symmetric amplification reaction was performed to provide the double-stranded target amplicon. Thus, according to one embodiment, step (a) comprises performing a symmetric amplification reaction to produce at least one double-stranded target amplicon. In this embodiment, the analytical sample does not contain or contains only minor amounts of the target strand as single-stranded amplicon. As is described in the introduction, a symmetric amplification has the advantage that it is more sensitive than an asymmetric amplification reaction. Therefore, it e.g. reliably enables the production of target amplicons from rare target nucleic acids.

Furthermore, the analytical sample comprises a compound (A), which is a water-soluble polyanionic co-polymer comprising maleic acid. As is shown by the examples, including compound (A) into the analytical sample is essential for the improvement that is achieved. The water-soluble polyanionic co-polymer comprising maleic acid can be added in form of the free acid or as salt. If desired, also two or more compounds (A) can be included in the analytical sample. Thus “a” compound (A) refers to at least one compound (A). As is shown by the examples, including a compound (A) as defined herein into the analytical sample greatly improves the obtained melting profiles. Compound (A) can be added in form of a solution or as dry matter. As is shown in the examples, it may also be added in a freeze-dried form. Preferably, compound (A) is added in form of a salt to prepare the analytical sample. E.g. compound (A) can be added as alkali metal salt, e.g. as sodium salt.

According to one embodiment, compound (A) is a water-soluble polyanionic co-polymer consisting of two monomeric species, wherein one monomeric species is maleic acid and the other monomeric species comprises at least one carboxyl group. Preferably, compound (A) is poly(acrylic acid-co-maleic acid) (PAMA). According to one embodiment, the poly(acrylic acid-co-maleic acid) used comprises acrylic acid and maleic acid in a molar ratio of 1:10 to 10:1, 1:5 to 5:1 or 1:2 to 2:1. Preferably, the poly(acrylic acid-co-maleic acid) used comprises acrylic acid and maleic acid in a molar ratio of 1:1.

According to one embodiment, compound (A) has an average molecular weight that lies in a range selected from 2,000 Da to 300,000 Da, 10,000 Da to 250,000 Da, 20,000 Da to 200,000 Da, 30,000 Da to 150,000 Da, 40,000 Da to 125,000 Da and 50,000 Da to 100,000 Da. Preferably, compound (A) has an average molecular weight that lies in a range of 25,000 Da to 100,000 Da, more preferred 35,000 Da to 75,000 Da. As described above, compound (A) is preferably PAMA.

According to one embodiment, the analytical sample comprises compound (A) in a concentration of at least 0.02% (w/v), at least 0.03% (w/v), at least 0.04% (w/v), at least 0.05% (w/v), at least 0.075% (w/v), at least 0.085% (w/v), at least 0.1% (w/v) or at least 0.15% (w/v). Suitable concentrations can also be determined by the skilled person. As is shown by the examples, already low amounts of compound (A) in the analytical sample to be analysed by melting curve analysis have a beneficial effect on the obtained melting profile. According to one embodiment, the analytical sample comprises compound (A) in a concentration that lies in the range of 0.02% (w/v) to 10% (w/v), 0.05% (w/v) to 7.5% (w/v), 0.075% (w/v) to 5% (w/v), 0.85% (w/v) to 4% (w/v), 0.1% (w/v) to 3% (w/v), 0.125% (w/v) to 2% (w/v), 0.15% to 1.5% (w/v), 0.25% to 1% (w/v) or 0.35% to 0.75% (w/v). As is shown by the examples, these ranges are particularly suitable when using PAMA as compound (A).

In certain embodiments, compound (A) may be already present during the amplification reaction and can e.g. be added prior to performing the amplification reaction. This has the advantage that it is not necessary to add compound (A) after the amplification reaction was performed, thereby saving intermediate handling steps. This embodiment is in particular suitable, if also the detection probe or detection probe set is also present during the amplification reaction as in this case a closed tube format can be used. However, care should be taken that the concentration of compound (A) is chosen such that the amplification reaction is not inhibited. According to a preferred embodiment, preparation of the analytical sample in step (a) comprises performing an amplification reaction in the absence of compound (A) to produce at least one double-stranded target amplicon and then adding compound (A) to the produced double-stranded target amplicon. Accordingly, in this embodiment, compound (A) is added after the amplification reaction was completed to prepare the analytical sample for melting curve analysis.

Furthermore, the analytical sample comprises at least one detection probe or at least one detection probe set capable of hybridizing to the target strand of the target amplicon. The first aspect of the present invention pertains to a probe-based melting curve analysis. This type of assay is based on the use of at least one detection probe or at least one detection probe set which upon hybridization form a double-stranded duplex with the target strand. The dissociation characteristics of the formed double-stranded duplex is then analysed to characterise the target nucleic acid. This type of assay is well-known to the skilled person and was explained in detail in the background of the invention to which it is referred. As described, different types of detection probes and detection probe sets can be used in a probe-based melting curve analysis. Non-limiting embodiments are again described in the following.

The term “detection probe” as used herein in particular refers to a probe that is used to detect the target amplicon. The detection probe is used to prove the presence of the target amplicon. This detection is independent from the oligonucleotides, which are used for the amplification itself. The detection probe is capable of hybridizing to the target strand under appropriate conditions. Usually, even though possible, the detection probe will not span the full length of the target strand of the amplicon but will hybridize to and thus span a certain region of the target strand, herein also referred to as “target region”. Therefore, preferably, the detection probe or the probes of the detection probe set span a target region of the target strand. According to one embodiment, the target region is a specific region of the target strand that is by the scope of the invention suspected to have nucleotide variation(s) and/or is of diagnostic relevance. Said target region may comprise e.g. a mutation, allelic variation or SNP. A detection probe set comprises two or more detection probes which hybridize in close proximity to each other to the target strand. The probes comprised in the detection probe set span together the target region.

According to one embodiment, the detection probe or probes comprised in the detection probe set is an oligonucleotide or polynucleotide. When referring to the probes, the terms “oligonucleotide” and “polynucleotide” are used interchangeably herein. Subsequently, we explain suitable designs by referring to the detection probe. The respective description likewise applies, however, to the probes comprised in the detection probe set if not indicated otherwise. An oligonucleotide or polynucleotide that may be used as detection probe may be composed of deoxyribonucleotides and/or ribonucleotides and may also comprise modified nucleotides and/or nucleotide analogues. The length of the detection probe may lie in a range selected from 10 to 200 nucleotides, 15 to 150 nucleotides, 20 to 100 nucleotides, 25 to 75 nucleotides and 25 to 50 nucleotides. Detection probes suitable for performing a probe-based melting curve analysis may be e.g. single-stranded, double-stranded or partially single- and double-stranded. Typical, but non-limiting examples of detection probes were described in the background of the invention and will also be described below.

In order to allow hybridization to the target strand, the at least one detection probe used is at least partially complementary to the target strand and thus anneals thereto under hybridization conditions. The term “complementary” in particular refers to the ability of two nucleotide sequences, such as the detection probe and the target strand to bind sequence-specifically to each other by hydrogen bonding through their purine and/or pyrimidine bases according to the usual Watson-Crick rules for forming a double-stranded duplex. Furthermore, obviously the term “complementary” also refers to the ability of nucleotide sequences that may include modified nucleotides or analogues of deoxyribonucleotides and ribonucleotides to bind sequence-specifically to each other by other than the usual Watson-Crick rules to form alternative double-stranded duplexes. In order to allow hybridization of the detection probe to the target strand, it is not necessary that they are 100% complementary. The complementarity required for hybridization also depends on the length of the formed duplex. However, it is preferred that the detection probe and the target strand are at least 80%, at least 90%, preferably at least 95%, at least 96%, at least 97%, at least 98% or at least 99% complementary. According to one embodiment, the number of mismatches between detection probe and target strand is 10 or less, 9 or less, 8 or less, 7 or less, 6 or less, 5 or less, 4 or less, 3 or less, 2 or less or 1 or there are no mismatches. As discussed above, the number of mismatches influences the Tₘ of the formed duplex and the formed melting curve. As used herein, the terms “hybridization” and “annealing” are used interchangeable, and in particular refer to the process by which two sequences complementary to each other (e.g. detection probe and target strand or target strand and complementary strand of the amplicon) bind together to form a double-stranded duplex. The term “duplex” in particular refers to a structure formed as a result of hybridization between two complementary sequences of nucleic acids. Such duplexes can be formed by the complementary binding of two DNA segments to each other, two RNA segments to each other, or of a DNA segment to an RNA segment, the latter structure being termed as a hybrid duplex. As described, either or both members of such duplexes can contain modified nucleotides and/or nucleotide analogues as well as nucleoside analogues.

In one embodiment of the invention, the detection probe or detection probe set is used in combination with a double stranded nucleic acid-specific dye such as an intercalating dye for the melting curve analysis. According to one embodiment, an unlabelled detection probe or detection probe set is used. When using an unlabelled detection probe or probe set, melting transitions of the double stranded target duplexes can be determined e.g. by monitoring fluorescence intensity of double stranded nucleic acid-specific (dsNAS) dyes. This format was also described in the background of the invention to which it is referred. In one embodiment, the double stranded nucleic acid-specific dye is an intercalating dye. It may be e.g. selected from the group consisting of SYBR® Green I₁ SYBR® Gold, ethidium bromide, propidium bromide, Pico Green, Hoechst 33258, YO-PRO-I and YO-YO-I, SYTO®9, LC Green®, LC Green® Plus+, EvaGreen™. According to one embodiment, the analytical sample comprises the dye in a saturating concentration. The saturating concentration is the concentration that provides the highest fluorescence intensity possible in the presence of a predetermined amount of double-strands. Because these dyes can be present at significantly higher concentrations without significantly interfering with certain nucleic acid reactions, these dyes are particularly useful for use in a melting curve analysis.

According to a preferred embodiment, the at least one detection probe or probes comprised in the at least one detection probe set are labelled. Preferably, the label is a reporter which allows monitoring the dissociation of the double-stranded duplexes during heating step (c).

Suitable labels include but are not limited to be applicable to absorption, fluorescence, chemiluminescece, colorimetric measurement, further electrochemical, voltametric, pH, amperometric, resistive, and capacitive measurement, even further to Raman, NMR, MEMS or radioactivity measurement.

According to a preferred embodiment, the detection probe or probes in the detection probe set are labeled with a fluorophore. Thus, according to a preferred embodiment the label is a fluorescent label. The label can be e.g. selected from the group of FAM (5- or 6-carboxyfluorescein), VIC, NED, Fluorescein, FITC, IRD-700/800, CY3, CY5, CY3.5, CY5.5, TAMRA, BODIPY TMR, Oregon Green, Rhodamine Green, Rhodamine Red, Texas Red, Alexa Fluor PET, Biosearch Blue™, Marina Blue®, Bothell Blue®, Alexa Fluor® 350, SYBR® Green 1, Fluorescein, EvaGreen™, Alexa Fluor® 488, JOE™, VIC™, HEX™, TET™, CAL Fluor® Gold 540, Yakima Yellow®, ROX™, CAL Fluor® Red 610, Alexa Fluor® 568, Quasar® 670, LightCycier Red640®, Alexa Fluor 633, Quasar® 705, LightCycler Red705®, Alexa Fluor® 680, SYTO®9, LC Green®, LC Green® Plus+, EvaGreen™.

The detection probe or one or more probes comprised in the detection probe set may e.g. be selected from the group consisting of TaqMan® probes, molecular beacon probes, scorpion probes, FRET probes or light cycler probes.

According to one embodiment, the detection probe or probes comprised in the detection probe set is or are labelled with a fluorophore and a quencher suitable to quench fluorescence of the fluorophore when the probe is not hybridized to the target strand. Respectively dual labelled probes are particularly preferred and different formats are available. Respective probes are e.g. commercially available as TaqMan® probes. As described above and as will be explained in further detail below, dissociation of respective probes from the target strand results in a decrease of fluorescence. The dual labelled probes preferably comprise a fluorophore and a quencher, wherein the quencher quenches the fluorescence emitted by the fluorophore when excited by a light source. Suitable quenchers include but are not limited to TAMRA, DABCYL, Black Hole Quencher (BHQ), Iowa Black or minor groove binders. A suitable pair may be for example the fluorophore FAM and the quencher TAMRA, but any other fluorophore/quencher pair may also be used. As long as the fluorophore and the quencher are in close proximity, quenching inhibits any fluorescence signals. Thus, in an unbound state no fluorescence or only a low fluorescence signal is emitted from the dual labelled probe. However, if the probe hybridizes to the target strand, quencher and fluorophore become spatially separated from one another. Excited by a light source, e.g. from a cycler, the fluorophore is then emitting light of a defined wavelength, and the emitted light can be detected thereby allowing to monitor the dissociation of the probe from the target strand. Within a dual labelled detection probe, the fluorophore may e.g. be positioned at the 5′ end and the quencher may be positioned at the 3′ end. Yet another alternative may be that within the dual labelled oligonucleotide probe fluorophore or quencher or both are positioned within the oligonucleotide sequence.

Molecular beacons are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one probe arm and a quencher is covalently linked to the end of the other probe arm. Molecular beacons do not fluoresce when they are free in solution. In the absence of targets, the probe is dark, because the stem places the fluorophore so close to the non-fluorescent quencher that they transiently share electrons, eliminating the ability of the fluorophore to fluoresce. However, when they hybridize to the target sequence they undergo a conformational change that enables them to fluoresce brightly. When the probe encounters the target strand, it forms a probe-target hybrid that is longer and more stable than the stem hybrid. The rigidity and length of the probe-target hybrid precludes the simultaneous existence of the stem hybrid. Consequently, the molecular beacon undergoes a spontaneous conformational reorganization that forces the stem hybrid to dissociate and the fluorophore and the quencher to move away from each other, thereby restoring fluorescence. Molecular beacons can be used that possess differently colored fluorophores, thereby enabling multiplexing analyses. This principle is well-known to the skilled person and thus, does not need any detailed description herein.

Scorpion primers (see e.g. Thelwell et al. (2000) Nucl Acid Res 28: 3752-61) are bi-functional molecules in which a primer is covalently linked to the probe. The molecules also contain a fluorophore and a quencher. In the absence of the target, the quencher nearly absorbs the fluorescence emitted by the fluorophore. During the Scorpion PCR reaction, in the presence of the target, the fluorophore and the quencher separate which leads to an increase in the fluorescence emitted. The fluorescence can be detected and measured in the reaction tube.

Also suitable for detection are FRET probe sets. A respective detection probe set usually comprise a pair of single-stranded labeled probes. Probe 1 (the donor probe) is labeled at its 3′-end with a donor fluorophore (e.g. fluorescein) and Probe 2 (the acceptor probe) is labeled at its 5′-end with one of e.g. four available fluorophores (e.g. red 610, 640, 670 or 705). If the probe is present during amplification, the free 3′ hydroxyl group of Probe 2 can be blocked, e.g. with a phosphate group (P) to prevent polymerase extension. If said probe or the whole detection probe set is added after the amplification reaction, a blockade of the free 3′ OH group is not necessary. Both probes of the detection probe set hybridize to the target strand in close proximity to each other. When hybridized to the target strand, the probes should be in close proximity, e.g. not more than 1 to 5 nt apart. Upon hybridization to the target strand, the donor dye comes into close proximity to the acceptor dye. When the donor dye is excited by light e.g. from the used light cycler instrument, energy is transferred by Fluorescence Resonance Energy Transfer (FRET) from the donor to the acceptor dye. The energy transfer causes the acceptor dye to emit light at a longer wavelength than the light emitted from the instrument. The acceptor fluorophore's emission wavelength can then be detected by the instrument's optical unit. Upon dissociation of the formed target duplex by heating, at least one of the probes is released which e.g. results in a decrease in fluorescence of the acceptor probe and an increase in fluorescence of the donor probe. This principle is well-known to the skilled person and thus, does not need any detailed description herein.

Of course other suitable designs for detection probes or detection probe sets can be used in conjunction with the present invention as long as they allow a probe-based melting curve analysis.

According to one embodiment, the analytical sample comprises a double-stranded nucleic acid specific dye in addition to the labeled detection probe or labeled detection probe set. As described above, the double-stranded nucleic acid specific dye can be selected from the group consisting of SYBR® Green I, SYBR® Gold, ethidium bromide, propidium bromide, Pico Green, EVAgreen, Hoechst 33258, YO-PRO-I and YO-YO-I. According to one embodiment, the double-stranded nucleic acid specific dye is spectrally distinguishable from the probe labels.

According to one embodiment, no double-stranded nucleic acid specific dye such as e.g. an intercalating dye is comprised in the analytical sample. In this embodiment at least one labeled detection probe or labeled detection probe set is used to allow performing a melting curve analysis.

According to one embodiment, a multiplex melting curve analysis is performed wherein two or more double-stranded duplexes are analysed at once. To prepare the analytical sample, a multiplex amplification reaction is performed in order to produce two or more different target amplicons, preferably in one amplification reaction. In this embodiment, the analytical sample comprises two or more different double-stranded target amplicons and optionally, corresponding target strands as single-stranded amplicons in case an asymmetric amplification is performed. In this embodiment, the analytical sample comprises for each target amplicon at least one detection probe or at least one detection probe set in order to allow detection of the different target amplicons in the same melting curve analysis. To allow a multiplex analysis, the detection probes or detection probe sets used for detecting the different target amplicons may comprise different labels in order to allow a distinction in the obtained melting profiles. However, they may also carry the same label. In this case, it is preferred that detection probes or detection probe sets carrying the same label form double-stranded target duplexes with their target strands which differ from each other in their melting temperature (Tₘ) in a way that they are distinguishable by melting curve analysis on a given instrument. Hence, the number of different target nucleic acids analyzable in parallel in a multiplexing approach and thus the number of different target amplicons analyzable in parallel in a multiplexing melting curve analysis follows inter alia from the number of different melting temperatures which can be distinguished from one another by the appropriate analytical instrument, combined with the number of the different fluorescent labels which can be distinguished from one another at different wavelengths by the particular analytical instrument. As described, if two double-stranded duplexes formed between the detection probe and the target strand have the same melting temperature, then these duplexes can nonetheless be specifically detected together and distinguished from one another in a multiplexing approach, if their own specific detection probes or probe sets have different fluorescent labels which emit the fluorescence at different wavelengths, so that these can be detected in different fluorescence channels. Conversely, detection probes which hybridize to different target strands can have the same fluorescent labeling if the melting temperatures of the formed target duplexes differ. These are then detected in the same fluorescence channel, but can nonetheless be distinguished from one another through their different melting temperatures.

The detection probe or detection probe set can be already present during the amplification reaction. This is also common in prior art melting curve analysis methods. Similarly as described above in conjunction with the addition of compound (A), this has the advantage, in particular if compound (A) is also present during the amplification reaction, that no additional handling steps are required after completion of the amplification reaction and before starting the melting curve analysis. This advantageously allows to use a closed tube format. Directly after completion of the amplification reaction, the melting curve analysis can be performed, i.e. without further processing steps to prepare the analytical sample for step (a). However, in particular when using dual labelled probes it was observed that the background fluorescence in the melting curve analysis can be reduced when the detection probe or detection probe set is added after completion of the amplification reaction. Therefore, according to one embodiment, preparation of the analytical sample in step (a) comprises performing an amplification reaction to produce at least one double-stranded target amplicon and adding the at least one detection probe or at least one detection probe set to the produced double-stranded target amplicon. According to one embodiment, preparation of the analytical sample in step (a) comprises performing an amplification reaction to produce at least one double-stranded target amplicon and adding compound (A) and the at least one detection probe or at least one detection probe set to the obtained amplification product which comprises the double-stranded target amplicon. Thus, preferably, compound (A) and the at least one detection probe or at least one detection probe set are added after performing the amplification reaction. According to one embodiment, the at least one detection probe or at least one detection probe set and compound (A) are comprised in a composition that is added to the amplification product after completion of the amplification reaction. The composition may have the form of a solution but may also have a solid form. As described in the examples, the detection probe or detection probe set and compound (A) may be provided in form of a composition such as e.g. a freeze-dried composition.

According to a preferred embodiment, a symmetric amplification reaction is performed to produce at least one double-stranded target amplicon. As described above, when performing a symmetric amplification, the analytical sample is substantially free of the target strand as single-stranded amplicon.

The analytical sample may comprise further additives in order to improve the melting curve analysis. According to one embodiment, the analytical sample additionally comprises betaine. As is shown by the examples, including betaine in addition to compound (A) in the analytical sample further improves the results of the melting curve analysis. However, betaine alone is not suitable to improve the melting curve profile, in particular when a symmetric amplification reaction is performed to produce the double-stranded target amplicon. Betaine may be present during the amplification reaction or may be added after completion of the amplification reaction. Betaine is a common additive for amplification reactions and may be present, e.g. in the amplification mixture. However, even if betaine is already present during amplification, further betaine may be added after the amplification reaction was completed. According to one embodiment, the analytical sample comprises betaine in a final concentration of ≦2 M, ≦1.5 M, ≦1.2 M, ≦1 M, ≦0.8 M, ≦0.6 M, ≦0.5 M≦0.4 M, ≦0.3 M or ≦0.25 M. According to one embodiment, betaine is not present in the analytical sample.

According to one embodiment, after completion of the amplification reaction, a composition comprising compound (A), at least one detection probe or detection probe set and betaine is added to prepare the analytical sample.

According to one embodiment, the analytical sample additionally comprises a chelating agent such as e.g. EDTA. EDTA is preferably added after completion of the amplification and may be e.g. comprised in a composition together with compound (A) and/or the detection probe or detection probe set. The concentration of EDTA in the analytical sample is preferably less than 20 mM, more preferred less than 15 mM.

It was found by the inventors that performing a nucleic acid isolation step after performing the amplification reaction significantly improves the obtained melting profiles, in particular if a symmetric amplification reaction was performed. Without wishing to be bound in theory, it is believed that after performing the amplification reaction, “melting inhibitors” are present in the amplification product that hamper the subsequent probe-based melting curve analysis. Respective inhibitors are apparently removed during nucleic acid purification as the target amplicons are provided in a pure form. However, performing a respective nucleic acid isolation step after completing the amplification and prior to performing the melting curve analysis is disadvantageous, as it increases the processing time, costs and poses the risk that target amplicon gets lost during the purification procedure. Therefore, it is preferred that no nucleic acid purification is performed after completion of the amplification reaction. Thus, preferably, the target amplicon is not purified or partially purified after amplification and prior to performing the actual melting curve analysis. As is shown by the examples, including compound (A) in the analytical sample allows to use unpurified amplification products for the probe-based melting curve analysis and the respective analytical samples provide strong, clear melting curve profiles even if a symmetric amplification was performed for providing the double-stranded target amplicon. Without wishing to be bound in theory, it is believed that compound (A) somehow counteracts or neutralizes melting inhibitors that are present in the unpurified amplification reaction.

If no purification is performed after completion of the amplification reaction, the analytical sample will accordingly comprise residual components of the amplification reaction such as e.g. residual primers, dNTPs, salt, polymerase and/or Mg ions.

After preparing the analytical sample, it can be analysed by performing a melting curve analysis. As described above, if compound (A) and the at least one detection probe or detection probe set are already present during the amplification reaction, the sample obtained after performing the amplification reaction directly provides the analytical sample. In this embodiment, it is not required to add further additives after performing the amplification reaction and the melting curve analysis can be directly started after amplification. A respective “closed tube format” is advantageous, as it reduces the required handling steps and reduces the risk of errors or contaminations during analysis. However, as described above, it is within the scope of the present invention and also preferred for many embodiments to add compound (A) after performing the amplification reaction. Preferably, also the at least one detection probe or at least one detection probe set is added after performing the amplification reaction.

### Step (b)

In step (b), a double-stranded target duplex comprising the detection probe or detection probe set hybridized to the target-strand is provided. Depending on the used embodiment, the double-stranded target duplex may already have been formed, at least partially, during the amplification reaction if the detection probe or detection probe set is already present during the amplification reaction.

However, as described above, it is preferred to add the at least one detection probe or at least one detection probe set after completion of the amplification reaction. As described above, after completion of the amplification reaction, the target strand is largely and in case of a symmetric amplification reaction even substantially exclusively comprised in the double-stranded target amplicon wherein it is hybridized to the complementary strand. To allow efficient formation of the double-stranded target duplex it is thus preferred to separate the double-stranded target amplicon comprised in the analytical sample in order to allow hybridization of the at least one detection probe or at least one detection probe set to its target strand. This is in particular important, if the analytical sample comprises a double-stranded target amplicon that was obtained in a symmetric amplification reaction as in this case, there is no excess single-stranded target strand.

According to one embodiment, step (b) comprises heating the analytical sample to a temperature wherein double-stranded molecules, in particular the double-stranded target amplicon, separate. Suitable denaturation temperatures depend inter alia on the length of the target amplicon. A commonly used denaturation temperature is e.g. at least 85° C., preferably at least 90° C. After denaturation, the temperature is reduced in order to allow annealing of the at least one detection probe or at least one detection probe set to the target strand, thereby allowing the double-stranded target duplex to form. The suitable annealing temperature again inter alia depends on the used detection probe or detection probe set. As described above, the GC content as well as the length of the formed target duplex has an influence on the annealing temperature. During this annealing step, a double-stranded target duplex comprising the detection probe or detection probe set is formed. These principles are well-known to the skilled person and thus, do not need any detailed description herein.

### Step (c)

In step (c), the analytical sample is gradually heated to obtain the melting profile. As described above, the analytical sample comprises the double-stranded target duplex. During the heating process, the dissociation of the double-stranded target duplex and accordingly, the separation of the at least one detection probe or detection probe set is measured. According to one embodiment, step (c) comprises heating the analytical sample in increments of 1° C. or less, 0.75° C. or less, 0.5° C. or less, 0.4° C. or less, 0.3° C. or less, 0.25° C. or less, 0.2° C. or less, 0.15° C. or less or 0.1° C. or less. The smaller the temperature increments the higher is the resolution in the melting profile. The temperature can be maintained at each temperature level for a certain holding time (e.g. 1 s to 15 sec). Measurement, respectively detection of dissociation can be performed continuously or at different temperatures, respectively temperature steps.

Preferably, the method according to the present invention is a fluorescence based melting curve analysis. In a fluorescence based melting curve analysis, the dissociation of the double-stranded target duplex during heating is measured based on changes in the emitted fluorescence. The analytical sample comprising the double-stranded target duplex is subjected to a stepwise increase in temperature, with fluorescence monitored continuously. Therefore, preferably, an instrument is used for performing the method according to the present invention that is configured for heating and cooling the analytical sample and furthermore, allows to monitor the emitted fluorescence. Suitable instruments such as light cyclers are well-known and available to the skilled person. Preferentially, melting curve analysis is performed using at least one labelled detection probe or at least one labelled detection probe set and a real time PCR cycler. Real-time PCR cyclers suitable for melting curve analysis are for example the Applied Biosystems 7500 Fast System and the 7900HT Fast Real-Time PCR System, Idaho Technology's LightScanner, Qiagen's Rotor-Gene instruments, and Roche's LightCycler 480 instruments. However, other cyclers may of course also be used.

According to a preferred embodiment, the fluorescence decreases when the detection probe or detection probe set dissociates from the target strand and accordingly, when the double-stranded target duplex is melted. This decrease is monitored. However, depending on the used detection probes and the used labels, also an increase in fluorescence can be monitored.

In a typical embodiment, the at least one detection probe or at least one detection probe set emits fluorescence under appropriate conditions when hybridized to the target strand. The fluorescence decreases when the detection probe or detection probe set dissociates from the target strand. According to one embodiment, after formation of the double-stranded target duplex the analytical sample is gradually heated e.g. to at least 80° C., for example with a transition rate of approximately 0.5° C./s or less, wherein the double-stranded target duplex melts again. Of course, also other transition rates may be used. Both target duplex formation and target duplex melting can be detected by continuously measuring the rate of fluorescence emitted in response to excitement by the cycler's light source. As described, the emitted fluorescence is determined and recorded during heating, thereby obtaining the data for the melting profile (melting curve). As described above, the obtained data can be represented by plotting fluorescence (F) over temperature (T), or, to make the analysis more convenient, can be represented by the negative first derivative (−dF/dT versus T). The melting temperature Tₘ of a double-stranded duplex can be determined. The melting temperature Tₘ is defined as the temperature at which 50% of the molecules are double-stranded and 50% are single-stranded. The melting temperature Tₘ can be derived e.g. from the inflection point of the fluorescence (F) versus temperature (T) curves, or the peak value of the −dF/dT versus T curve.

As described above, a peak within the melting curve indicates presence of a nucleic acid in the sample. Alternatively to the data projection in form of melting curves, the melting analysis raw data may of course be projected and displayed in any other format known in the prior art of statistical data presentation. According to one embodiment, target nucleic acids are not only detected in a qualitative manner, in which presence or absence of a target nucleic acid in a sample is determined, the method of the invention may also be used for semi-quantitative target nucleic acid detection, as the area under the curve, AUC of a melting peak is proportional to the amount of nucleic acid present in a sample. Thus, the melting curve analysis may be used for a quantitative assessment of a target nucleic acid present in a sample, e.g. a DNA amplicon in a symmetric PCR reaction.

Furthermore, as described above, it is also possible to perform a fluorescence based melting curve analysis by making use of specific dyes such as intercalating dyes. Suitable embodiments were described above. The respective principle is also well-known in the art and therefore, does not need any further description.

According to a preferred embodiment, the method is a fluorescence based melting curve analysis, comprising
(a) preparing an analytical sample by performing an amplification reaction to produce at least one double-stranded target amplicon, wherein preferably, the amplification reaction is a symmetric polymerase chain reaction, and adding poly(acrylic acid-co-maleic acid) and at least one detection probe or at least one detection probe set to the produced double-stranded target amplicon, thereby providing an analytical sample which comprises
   (i) at least one double-stranded target amplicon comprising a target strand and a complementary strand;
   (ii) at least one detection probe or at least one detection probe set capable of hybridizing to the target strand of the target amplicon and
   (iii) a compound (A), which is poly(acrylic acid-co-maleic acid);
   and wherein the analytical sample optionally comprises additionally the target strand as single-stranded amplicon;
(b) forming a double-stranded target duplex comprising the detection probe or detection probe set hybridized to the target strand, wherein a fluorescent signal is emitted when the double-stranded duplex is formed;
(c) gradually heating the analytical sample and measuring the dissociation of the double-stranded target duplex during heating by recording the decrease in fluorescence.

According to one embodiment, the method according to the present invention is performed using a “lab-on-a-chip” (LoC) system. “Lab-on-a-chip” generally stands for the idea of the scaling single or multiple lab processes, e.g. nucleic acid amplification and detection assays, to chip-format. As such, a “lab-on-a-chip” device may integrate one or several laboratory functions on a single chip of only millimeters to a few square centimeters in size. One advantage of such devices is that they allow the handling of extremely small fluid volumes in microfluidic systems. In general, microfluidic systems are advantageous as they do not require the discontinuation of a reaction when the addition of a compound is required. The possibility to add a reaction compound without interrupting the reaction process, in particular without the need for opening and closing the reaction vessel allows faster workflow and reduces the risk of contamination.

The most elaborated LoC systems are capable to process e.g. a diagnostic test from sample to result. Thus, systems are available, wherein the analytical method is carried out in a miniaturized system, e.g. a cartridge also referred to as LoC cartridge. Respective cartridges are e.g. described in WO2006/071770, US2009/0130658, WO 2006/042734 and DE 10 2008 004 646. The respective cartridges that are often used in LoC systems comprise the reagents necessary for performing the analytical method of interest in a dry form, preferably a freeze-dried form. Dry compositions of reagents are widely used in analytical methods, in particular in amplification reactions such as e.g. the polymerase chain reaction or for detecting other analytes such as proteins. Respective dry compositions usually comprise one or more or even all reagents necessary for the amplification. The use of respective dry compositions, in particular freeze-dried compositions, has the advantage that the dry compositions are stable during storage and therefore, respective freeze-dried compositions are often used in cartridges to provide all reagents necessary for the analysis method to be performed in the cartridge. Providing the reagents in a respective dry form has the advantage that the customer does not need to combine the necessary reagents himself. Instead, only a pre-determined amount of liquid such as water or a suitable buffer is added to reconstitute the dry reagents, thereby providing a reaction mix that is suitable for performing the intended amplification once the sample comprising the target nucleic acids is incorporated. In this setting, the composition according to the present invention is particularly advantageous. E.g. an amplification reaction can be performed in an amplification chamber of a LoC cartridge which comprises the necessary reagents for amplification. Furthermore, the LoC cartridge comprises a composition comprising compound (A) and preferably, at least one detection probe or detection probe set in a chamber of a LoC cartridge that is suitable for melting curve analysis. Preferably, the composition is a freeze-dried composition according to the third aspect of the present invention. After completion of the amplification reaction in the LoC cartridge, the liquid amplification reaction can be contacted with the freeze-dried composition comprising compound (A) and at least one detection probe or detection probe set which is thereby reconstituted. Thereby, the analytical sample is prepared and is ready for melting curve analysis in the LoC cartridge.

In a second aspect, the present invention provides a method for amplifying and detecting a target nucleic acid, comprising amplifying a target sequence of the target nucleic acid thereby providing a double-stranded target amplicon and performing a probe-based melting curve analysis as defined in the first aspect of the present invention. Suitable and preferred embodiments of the melting curve analysis according to the first aspect were described above and it is referred to the respective disclosure. As was also described above, the method according to the present invention allows the reliable analysis of asymmetric as well as of symmetric amplification products.

According to one embodiment, an amplification mixture is set up for amplifying the target sequence. The term “amplification mixture” as used herein in particular refers to a mixture of components necessary to amplify at least one target amplicon from a nucleic acid template. The mixture may e.g. comprise nucleotides (dNTPs), a thermostable polymerase, primers, and nucleic acids. The mixture may further comprise a buffer such as a Tris buffer, a monovalent salt and Mg ions. Suitable amplification mixtures are well-known to the skilled person. The concentration of each component is also well known in the art and can be further optimized by an ordinary skilled artisan.

Any source of nucleic acid, in purified or non-purified form, can be utilized as the starting nucleic acid for amplification. The nucleic acid may have been obtained from different types of biological samples such as e.g. body fluids in general, whole blood, serum, plasma, red blood cells, white blood cells, buffy coat; swabs, including but not limited to buccal swabs, throat swabs, vaginal swabs, urethral swabs, cervical swabs, throat swabs, rectal swabs, lesion swabs, abcess swabs, nasopharyngeal swabs and anal swabs, urine, sputum, saliva, semen, lymphatic fluid, liquor, amniotic fluid, cerebrospinal fluid, peritoneal effusions, feces, pleural effusions, fluid from cysts, synovial fluid, vitreous humor; aqueous humor, bursa fluid, eye washes, eye aspirates, pulmonary lavage, lung aspirates, tissues, including but not limited to, liver, spleen, kidney, lung, intestine, brain, heart, muscle, pancreas, tumor tissue, biopsis and cell cultures, bacteria, microorganisms, viruses, plants, fungi including samples that derive from the foregoing or comprise the foregoing. Materials obtained from clinical or forensic settings or environmental samples such as soil that contain or are suspected to contain target nucleic acids can also be used as starting material. Furthermore, the skilled artisan will appreciate that extracts, or materials or portions thereof obtained from any of the above exemplary samples can also be used as source for the nucleic acids. Preferably, the source or sample from which the nucleic acids to be analysed are obtained is derived from a human, animal, plant, bacteria or fungi. Preferably, the sample is selected from the group consisting of cells, tissue, bacteria, viruses and body fluids such as for example blood, blood products such as buffy coat, plasma and serum, urine, liquor, sputum, stool, CSF and sperm, epithelial swabs, vaginal swabs, cervix samples, biopsies, bone marrow samples and tissue samples, preferably organ tissue samples such as lung and liver or tumor tissue. The sample may be stabilized. Certain samples such as blood samples are usually stabilised upon collection, e.g. by contacting them with a stabilizer such as an anticoagulant in case of blood and samples derived from blood.

Prior to performing the amplification reaction, nucleic acids can be released from the sample if necessary, e.g. using appropriate lysing procedures. Lysis methods are well-known to the skilled person and thus, do not need any detailed description. According to one embodiment, the obtained lysate is directly used in the nucleic acid amplification reaction. This is feasible, if a lysis method is used which allows a direct amplification of the nucleic acids comprised in the lysate without prior purification of the nucleic acids. Such lysis buffers are e.g. described in PCT/EP2012/004632 and US 2011/0177516. Furthermore, the nucleic acids can first be isolated and purified prior to performing the nucleic acid amplification to provide the target amplicon(s). Methods for isolating nucleic acids are well-known in the prior art and therefore, do not need any detailed description here.

RNA or DNA can be used as template material for amplification. Accordingly, the target nucleic acid may be DNA or RNA. RNA is preferably first reverse transcribed into cDNA prior to amplification. According to one embodiment, the amplification employs DNA as template. The DNA may be single-stranded or double-stranded. In addition, a DNA-RNA hybrid which contains one strand of each may be utilized. A mixture of any of these nucleic acids may also be employed, or the nucleic acids produced from a previous amplification reaction using the same or different primers may be utilized. The target sequence to be amplified to provide the target amplicon may be only a region within a larger nucleic acid molecule or can be present initially as a discrete molecule, so that the target sequence that is amplified constitutes the entire target nucleic acid. It is not necessary that the target sequence to be amplified be present initially in a pure form; it may be a minor fraction of a complex mixture such as a lysis mixture or nucleic acid within a complex mixture of different nucleic acids. The nucleic acids subjected to the amplification reaction may contain more than one desired target nucleic acid. Furthermore, also different target sequences within a target nucleic acid molecule can be amplified to produce different target amplicons. Therefore, the method is useful for amplifying simultaneously multiple target sequences located on the same or different nucleic acid molecules. The nucleic acid(s) used for amplification may be obtained from any source.

Preferably, locus-specific primers are used for amplification. Methods for designing and obtaining primers are well-known to the skilled person and thus, do not need any detailed description here. E.g. oligonucleotide primers may be prepared using any suitable method, such as, for example, the phosphotriester and phosphodiester methods or automated embodiments thereof. Preferred primers have a length of from about 15-100, more preferably about 20-50, most preferably about 20-40 bases. They may comprise modified nucleotides or nucleotide analogs. By using appropriately designed primers, a target amplicon is produced which encompasses the target region that is detected by the at least one detection probe or detection probe set. Usually, the target sequence that is amplified to produce the target amplicon is larger than the target region that is detected by the detection probe or detection probe set. However, the target strand comprised in the target amplicon may also consist of the target region. As described above, the target region may e.g. be a region which comprises or is suspected to comprise sequence variations such as allelic variations, mutations or SNPs or which can be used to identify a certain target, such as e.g. a pathogen nucleic acid.

The amplicon size usually depends on the used amplification and furthermore, the intended analysis. These principles are well-known to the skilled person and thus, do not need any detailed description herein. According to one embodiment, the amplicon has a size selected from 40 to 1000 bp, 50 to 750 bp, 60 to 500 bp, 75 bp to 400 bp and 100 bp to 300 bp.

Suitable conditions for performing various kinds of amplification reactions are well-known to the skilled person and thus, do not need any detailed description here. If the target nucleic acid to be amplified is double-stranded, the strands of the nucleic acid are first separated to provide the template single-stranded, either as a separate step or simultaneously with the synthesis of the primer extension products. This strand separation can be accomplished by any suitable denaturing method including physical, chemical, or enzymatic means. One physical method of separating the strands of the nucleic acid involves heating the nucleic acid until it is completely (>99 percent) denatured. Typical heat denaturation may involve temperatures ranging from about 80° C. to 105° C., preferably about 90° C. to about 98° C. for times ranging from about 1 second to 10 minutes. This is e.g. the case in a polymerase chain reaction. In the case of isothermal amplification the strand separation may also be induced by an enzyme from the class of enzymes known as helicases or the enzyme RecA, which has helicase activity and is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), and techniques for using RecA are reviewed in C. Radding, Ann. Rev. Genetics, 16:405-37 (1982), which is hereby incorporated by reference.

As described above, when using the teachings of the present invention it is not necessary to perform a purification step after amplification. The obtained amplification product can be directly used in the melting curve analysis as described above. It is referred to the above disclosure which also applies here.

The method is in particular suitable in the field of basic research but also and in particular in the medical and diagnostic field. It can be e.g. used in order to detect the presence or absence of a pathogen in a sample, to detect and/or categorize genetic mutations, in particular single nucleotide polymorphisms (SNPs), SNP genotyping, tumor typing, to identify new genetic variants without sequencing (gene scanning), to determine the genetic variation in a population (for example viral diversity) prior to sequencing, mutation discovery (gene scanning), heterozygosity screening, DNA finger printing, haplotype blocks characterization, DNA methylation analysis, DNA mapping, species identification, pathogen detection, viral/bacterial population diversity investigation and HLA compatibility typing.

In a third aspect, a composition is provided which comprises at least one detection probe or at least one detection probe set and a compound (A) which is a water-soluble polyanionic co-polymer comprising maleic acid. As described above in conjunction with the method according to the first aspect, a respective composition can be added e.g. to an amplification product in order to set up an analytical sample for a probe-based melting curve analysis.

Preferably, compound (A) is a water-soluble polyanionic co-polymer consisting of two monomeric species, wherein one monomeric species is maleic acid and the other monomeric species comprises a carboxyl group. Most preferred, compound (A) is poly(acrylic acid-co-maleic acid). Suitable embodiments were described above in conjunction with the method according to the first aspect. Preferably, compound (A) is comprised in form of a salt.

Suitable and preferred embodiments for the detection probe or the detection probe set were also described above in conjunction with the method according to the first aspect of the invention. It is referred to the above disclosure.

According to one embodiment, the composition additionally comprises betaine. As described above, including betaine in addition to compound (A) in the analytical sample further improves the results. According to one embodiment, the composition does not comprise betaine. Furthermore, the composition may comprise a chelating agent.

The composition can be provided in liquid form, e.g. as aqueous solution. Furthermore, it may be provided in dried form and thus is a dried composition. According to one embodiment, the composition is a freeze-dried composition. As is shown by the examples, the composition according to the present invention can be provided as freeze-dried product, e.g. as lyophilized product. The lyophilized product may have any shape. According to one embodiment, a lyophilisate is provided using the method as described in WO2011/124667, herein incorporated by reference. In this embodiment, the composition is provided as spherical solid body which can be easily added to the amplification product to set up the analytical sample for a probe-based melting curve analysis. Respective freeze dried compositions are particularly suitable and beneficial e.g. for kit formats and “lab-on-a chip” (LoC) systems (see above).

According to one embodiment, the composition comprises at least one double-stranded target amplicon. In this embodiment, the composition refers to the analytical sample. As described above, the technology according to the present invention allows to obtain melting curve profiles from amplification products that were obtained in an asymmetric as well as in a symmetric amplification reaction. According to one embodiment, the double-stranded target amplicon was obtained in a symmetric amplification reaction and accordingly, the composition is substantially free of singe-stranded amplicons of the target strand. Furthermore, as described above, the present invention allows to process unpurified amplification products. Therefore, the composition may comprise reagents that were used/present in the amplification reaction such as e.g. a polymerase, primers, dNTPs, salts and/or ions.

According to a fourth aspect, the present invention provides a kit for performing a probe-based melting curve analysis, wherein the kit comprises at least one detection probe or at least one detection probe set and a compound (A) which is a water-soluble polyanionic co-polymer comprising maleic acid. Said kit can be advantageously used in order to prepare the analytical sample for a probe-based melting curve analysis. Thus, the kit may be used in a method according to the first or second aspect of the present invention. Optionally, the kit may comprise further reagents such as e.g. one or more reagents for performing an amplification reaction. E.g. the kit may additionally comprise a polymerase, a reaction mixture for performing an amplification reaction, such as preferably a polymerase chain reaction, dNTPs and/or primers.

Preferably, compound (A) comprised in the kit is a water-soluble polyanionic co-polymer consisting of two monomeric species, wherein one monomeric species is maleic acid and the other monomeric species comprises a carboxyl group. Most preferred compound (A) is poly(acrylic acid-co-maleic acid). Suitable and preferred embodiments were also described above in conjunction with the method according to the first aspect and it is referred to the respective disclosure. Preferably, compound (A) is provided in form of a salt.

The at least one detection probe or at least one detection probe set may be comprised in the kit in form of a single composition or can be provided separately in the kit. According to a preferred embodiment, the kit comprises a composition according to the third aspect of the present invention which was described in detail above. It is thus referred to the above disclosure.

According to a fifth aspect, the present invention pertains to the use of a compound (A) which is a water-soluble polyanionic co-polymer comprising maleic acid for preparing an analytical sample for melting curve analysis. As was explained above and as is shown by the examples, including a compound (A) into the analytical sample significantly improves the results of the performed melting curve analysis. The analytical sample was described in detail above in conjunction with the method according to the first aspect. The analytical sample comprises at least one double-stranded target amplicon comprising a target strand and a complementary strand. According to one embodiment, the analytical sample does not comprise or comprises only minor amounts of the target strand in a single-stranded form. As described above, this can be achieved by performing a symmetric amplification reaction to produce the double-stranded target amplicon. Furthermore, preferably, the analytical sample additionally comprises at least one detection probe or at least one detection probe set capable of hybridizing to the target strand of the target amplicon. Thus, according to one embodiment, the melting curve analysis is a probe-based melting curve analysis, in particular a fluorescence based melting curve analysis. Furthermore, the analytical sample may comprise betaine. Further details of the analytical sample and suitable and preferred embodiments and concentrations of the individual components were described above in conjunction with the method according to the first aspect and it is referred to the above disclosure for details. Preferably, compound (A) is a water-soluble polyanionic co-polymer consisting of two monomeric species, wherein one monomeric species is maleic acid and the other monomeric species comprises a carboxyl group. Most preferred compound (A) is poly(acrylic acid-co-maleic acid). Suitable and preferred embodiments were also described above in conjunction with the method according to the first aspect and it is referred to the respective disclosure. Preferably, compound (A) is added in form of a salt.

Numeric ranges described herein are inclusive of the numbers defining the range. The headings provided herein are not limitations of the various aspects or embodiments of this invention which can be read by reference to the specification as a whole. The term “acid” as used herein also refers to the salts of the respective acid, i.e. to the deprotonated form of the acid and vice versa. Preferably, preferred embodiments as described herein are used in combination with each other. Furthermore, embodiments that are described herein as comprising a certain subject-matter or steps according to one embodiment may also consist or substantially consist of the respective subject-matter or steps.

The present invention will now be described in further detail by the following non-limiting examples.

### EXAMPLES

### Example 1

### Melting Curve Analysis with a Dual Labelled Oligonucleotide Probe is Effective with Asymmetric PCR Products but not with Symmetric PCR Products

A symmetric and an asymmetric PCR reaction were set up for amplifying Corynebacterium glutamicum genomic DNA as target nucleic acid. The symmetric PCR reaction comprised the forward and reverse primers in an equal ratio (1:1) while the ratio was 1:3 in the asymmetric PCR. Following PCR amplification in a QuantiFast Probe PCR Mastermix (QIAGEN), a target amplicon specific dual-labelled probe (final concentration 83 nM) was added and the obtained analytical samples were subjected to melting curve analysis on a Real-time PCR cycler. The analytical sample was heated 5 min to 95° C., cooled down to 50° C. and then heated from 50° C. to 90° C. in temperature increments of 0.5° C., with 10 sec holding time per temperature increment/step. Fluorescence melting peaks were derived from the initial fluorescence (F) versus temperature (T) curves by plotting the negative derivative of fluorescence over temperature versus temperature (−dF/dT versus T).

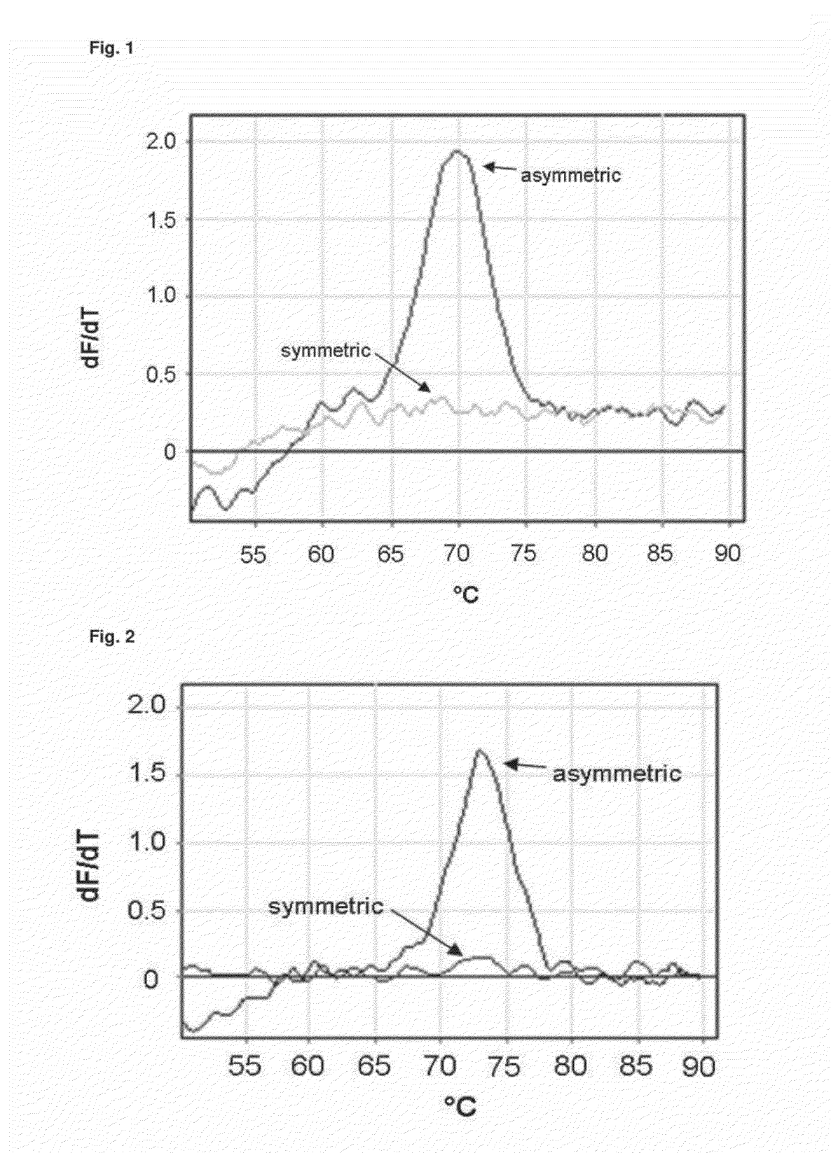

FIG. 1 shows the melting curve profiles obtained from symmetric and asymmetric PCR products. While the melting curve obtained in the analysis of the asymmetric PCR product displayed a concrete and well defined peak at approx. 70° C. (see FIG. 1, black curve), no such melting peak was obtained from the symmetric PCR product (see FIG. 1, light grey curve). Thus, no meaningful melting curve analysis was possible with the unpurified symmetric PCR product.

Furthermore, it was tested whether a removal of Mg²⁺ ions from the analytical sample by addition of EDTA to the crude amplification product could improve the results (91 mM EDTA in the analytical sample). The results are shown in FIG. 2. No improvement was seen and a meaningful melting profile with a sharp peak was only obtained from the analytical sample containing the amplification product that was obtained in an asymmetric PCR reaction.

### Example 2

### Addition of PAMA Greatly Improves Melting Performance of Symmetric PCR Amplicons

In previous experiments the inventors found that symmetric PCR reactions may be subjected to melting curve analysis using a dual labelled oligonucleotide probe when the amplification products were at least partially purified after amplification and prior to analysis. However, purification in general requires additional handling steps that are tedious, increase costs and extend the overall operating time. A minimum of handling steps is beneficial for automation, especially for “lab-on-a-chip” applications.

Therefore, the aim was to find a protocol which allows to avoid an additional purification step and still allows a reliable analysis of amplification products by melting curve analysis, irrespective of whether the amplification products were obtained in an asymmetric or symmetric amplification reaction. Therefore, a number of different additives were tested for their potential to improve the results of the melting curve analysis.

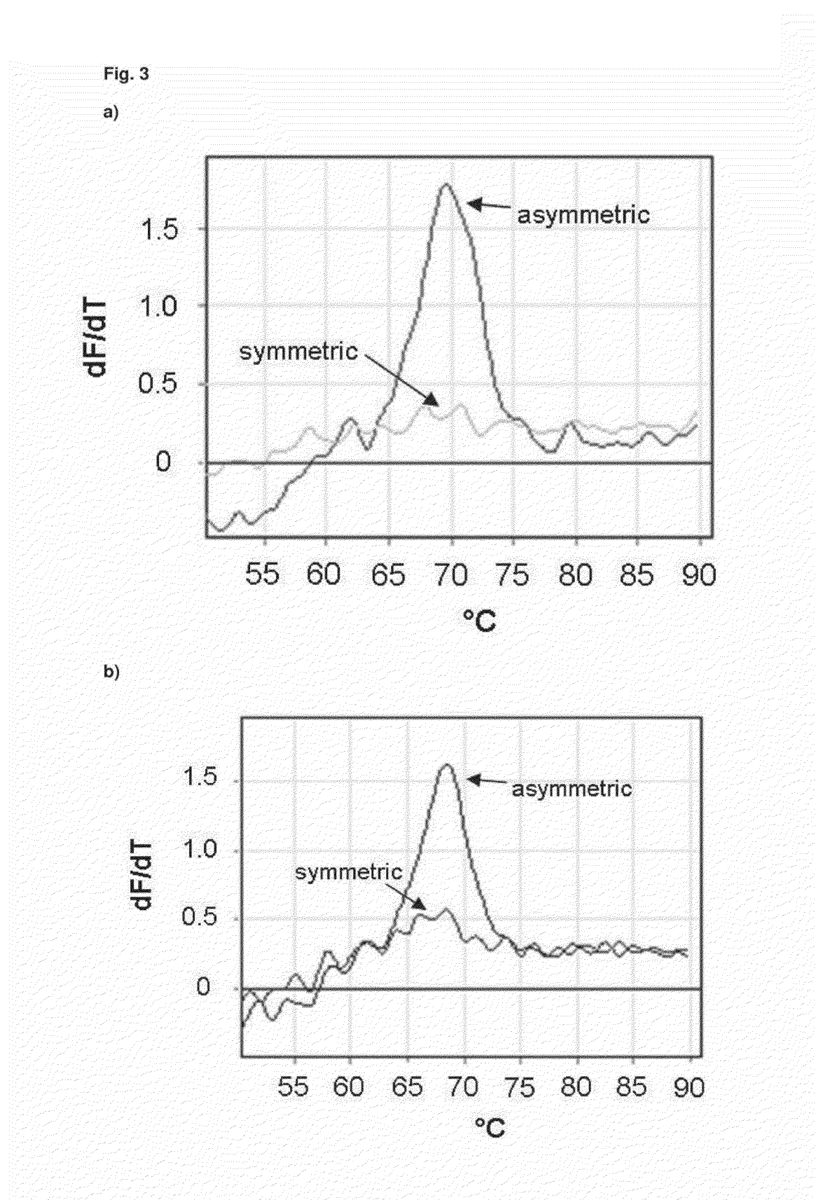

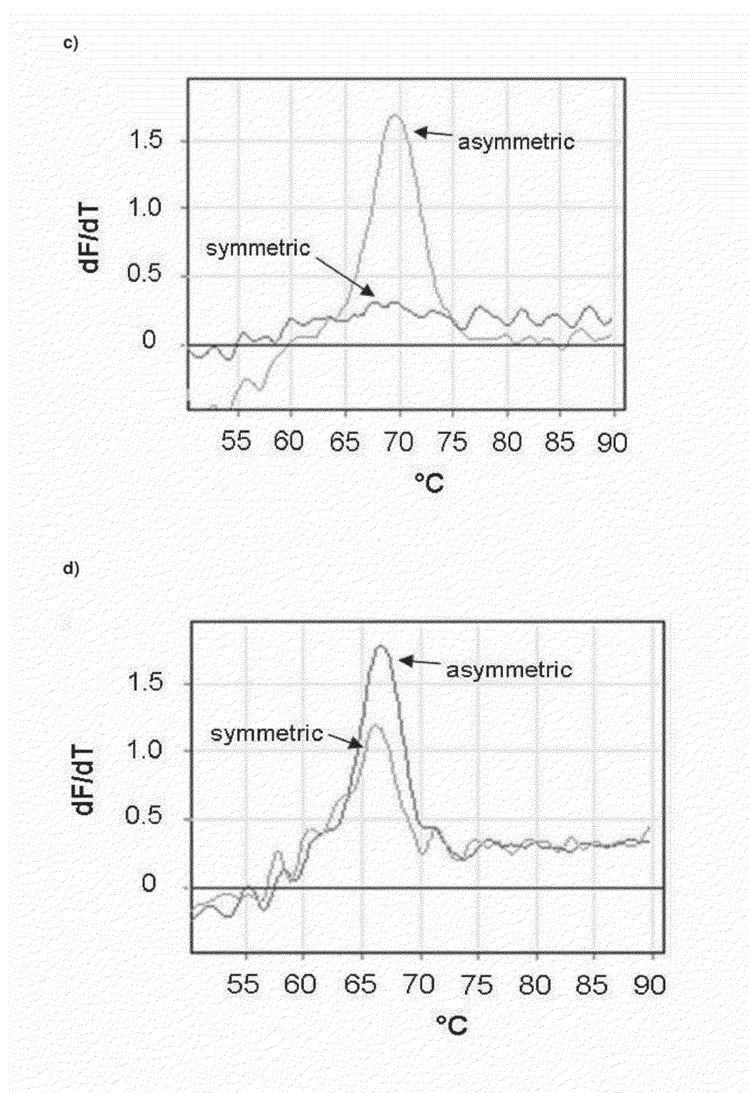

Asymmetric and symmetric PCR reactions were performed as described in example 1. After amplification either (a) water, (b) 0.05% (w/v) polyacrylic acid, (c) 0.1% (w/v) polyvinylpyrrolidone K15 10000 (PVP) or (d) 0.1% (w/v) of a sodium salt of poly (acrylic acid-co-maleic acid) (PAMA) were added to set up the analytical sample and the melting curves were determined as described in example 1. The above mentioned concentrations of the additives refer to the final concentration in the analytical sample. FIG. 3 shows the obtained melting profiles. Each panel depicts the obtained melting profile of an asymmetric PCR product and a symmetric PCR product. Wherein analytical samples comprising asymmetric PCR products showed clear melting peaks under all conditions tested, analytical samples comprising symmetric PCR products showed clear melting peaks only if the analytical sample comprised PAMA. Hence, including PAMA into the analytical sample renders also symmetric PCR reactions well suitable for melting curve analysis. No prior purification of the amplification product is necessary. Furthermore, the peak obtained for the asymmetric product was more narrow and thus better defined.

### Example 3

### Addition of a Combination of PAMA with Betaine and Optionally EDTA Further Improves Melting Performance

Symmetric and asymmetric PCR reactions were performed as described in example 1. After completion of the amplification reaction, a dual labelled detection probe (see example 1) and (a) water or (b) 0.09% (w/v) PAMA, 0.9 M betaine and 0.9 mM EDTA were added to set up the analytical sample.

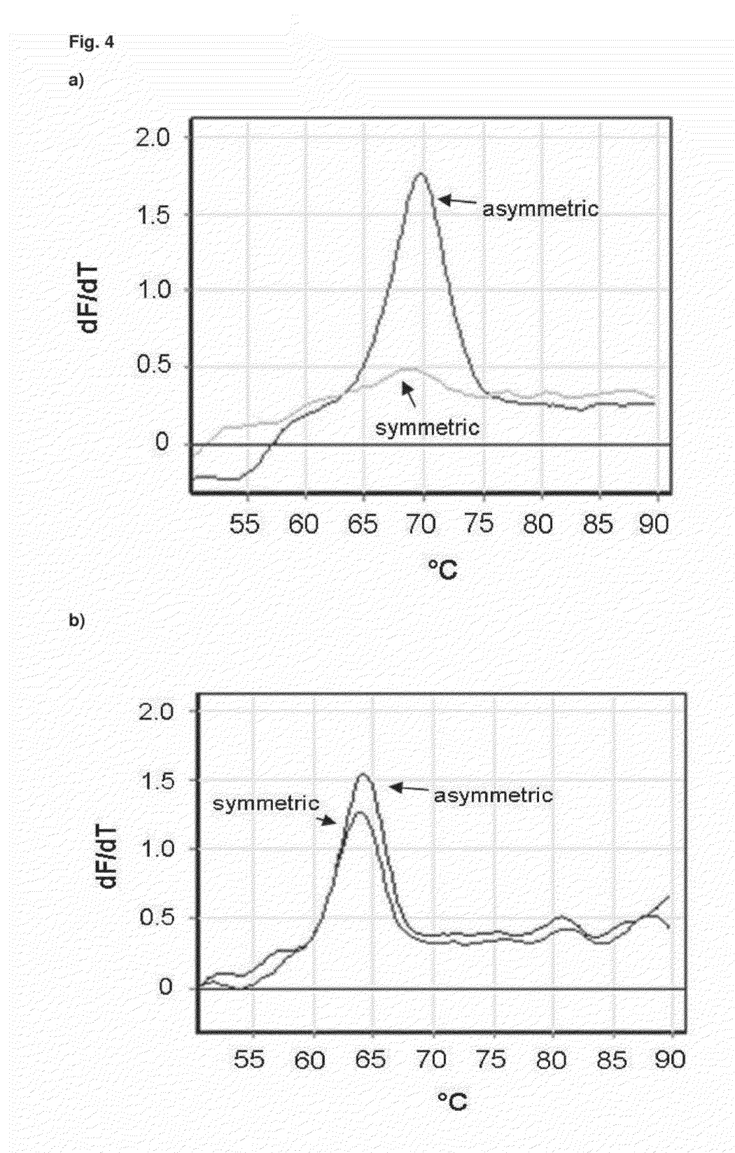

FIG. 4 shows the results. While the analytical sample comprising the product of a symmetric PCR reaction and water did not provide a meaningful melting curve (see FIG. 4a), the analytical sample comprising the product of a symmetric PCR reaction and PAMA, betaine and EDTA provided a concrete and well defined peak (see FIG. 4b).

Thus, the addition of the specifically selected substances substantially improved the melting curve performance of symmetric PCR products with dual labelled oligonucleotide probes.

### Example 4

### PAMA Improves Melting Performance of Symmetric PCR Reactions in a Variety of PCR Buffer Systems

PCR reaction buffers can vary in their composition. Therefore, it was tested whether the inclusion of PAMA in the analytical sample comprising amplification products obtained using different PCR reaction buffers produced the same beneficial results.

Symmetric PCR reactions were assembled for amplification of Corynebacterium glutamicum genomic DNA. The following QIAGEN mastermixes were used: QuantiFast Probe PCR Mastermix, QuantiFast Multiplex Mastermix, QuantiTect Virus Mastermix, QuantiTect Probe PCR Mastermix, QuantiTect Multiplex Mastermix and HotStarTaq Mastermix.

After completion of the amplification reaction in the various buffer systems, the analytical samples were set up by adding an amplicon-specific dual labelled probe (final concentration 100 nM), and (a) 667 mM betaine, 0.067% (w/v) PAMA and 0.67 mM EDTA (all final concentrations in the analytical sample), or (b) water, or (c) nothing (control). A melting curve analysis was performed as described in example 1. Each condition was tested in duplicate.

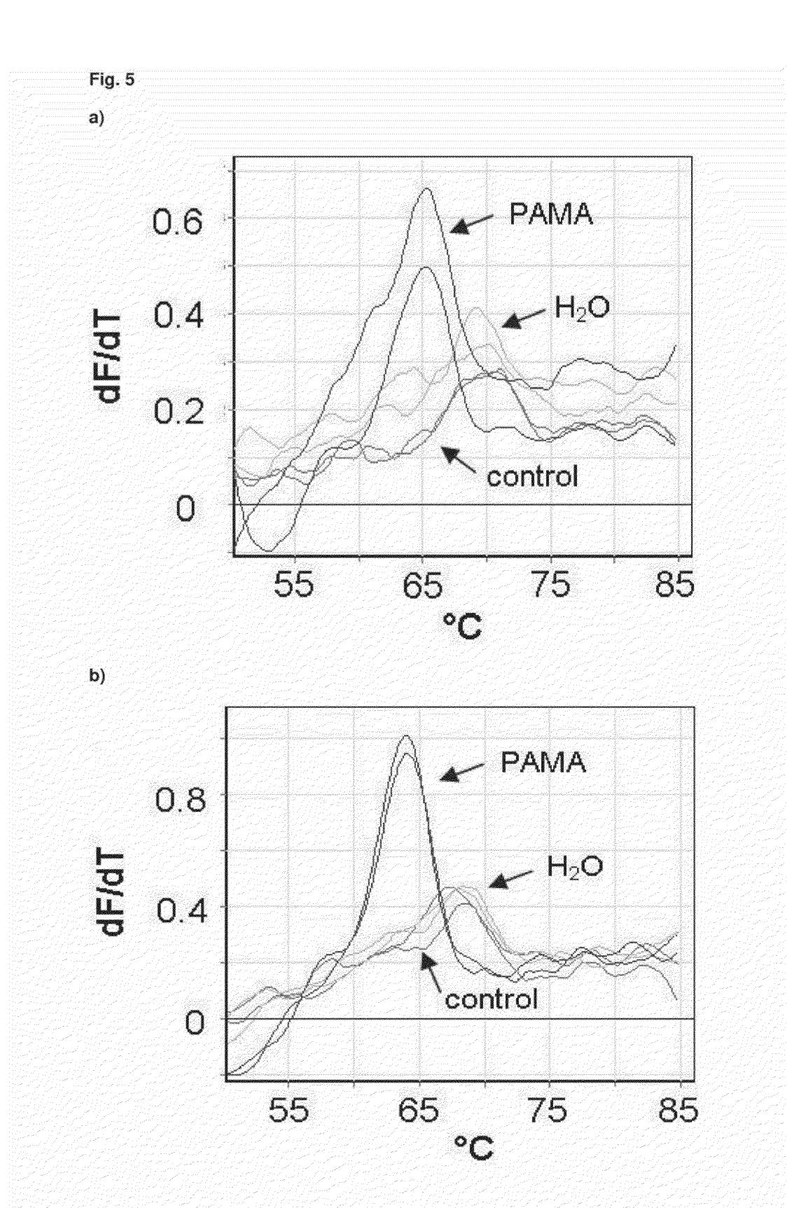

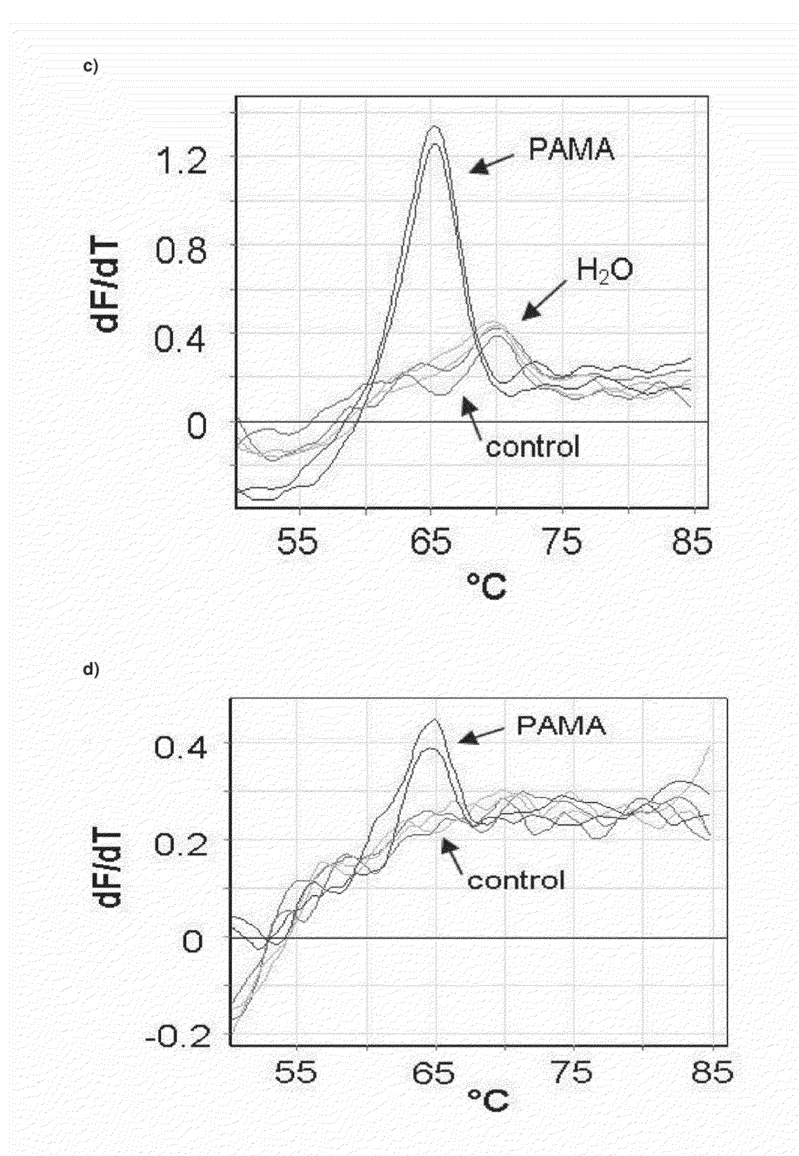

FIG. 5 shows the melting curves of analytical samples which were prepared from unpurified symmetric PCR reactions performed in various PCR buffer systems from QIAGEN. The analytical samples comprising PAMA provided clear, sharp melting profiles under all conditions tested, while the analytical samples without PAMA did not provide meaningful melting profiles. Thus, in all PCR reaction systems tested, addition of PAMA, EDTA and betaine significantly improved the melting performance with the dual labelled oligonucleotide probe. This demonstrates that PAMA is universally applicable to improve the melting curve analysis.

### Example 5

### Analysis of Different PAMA Concentrations

The following experiments were performed in order to determine the optimal PAMA concentration range, and furthermore, to investigate the influence of betaine and EDTA on the melting curve performance using dual labelled oligonucleotide probes.

Symmetric PCR reactions were assembled for amplification of Corynebacterium glutamicum genomic DNA in QuantiFast Multiplex Mastermix. Following PCR amplification, the PCR reactions were supplemented with 0-0.5% PAMA, 100 nM probe and 250 mM betaine (leading to 390 mM final concentration of betaine) to set up the analytical sample (final concentrations). Melting curves were obtained as described in example 1.

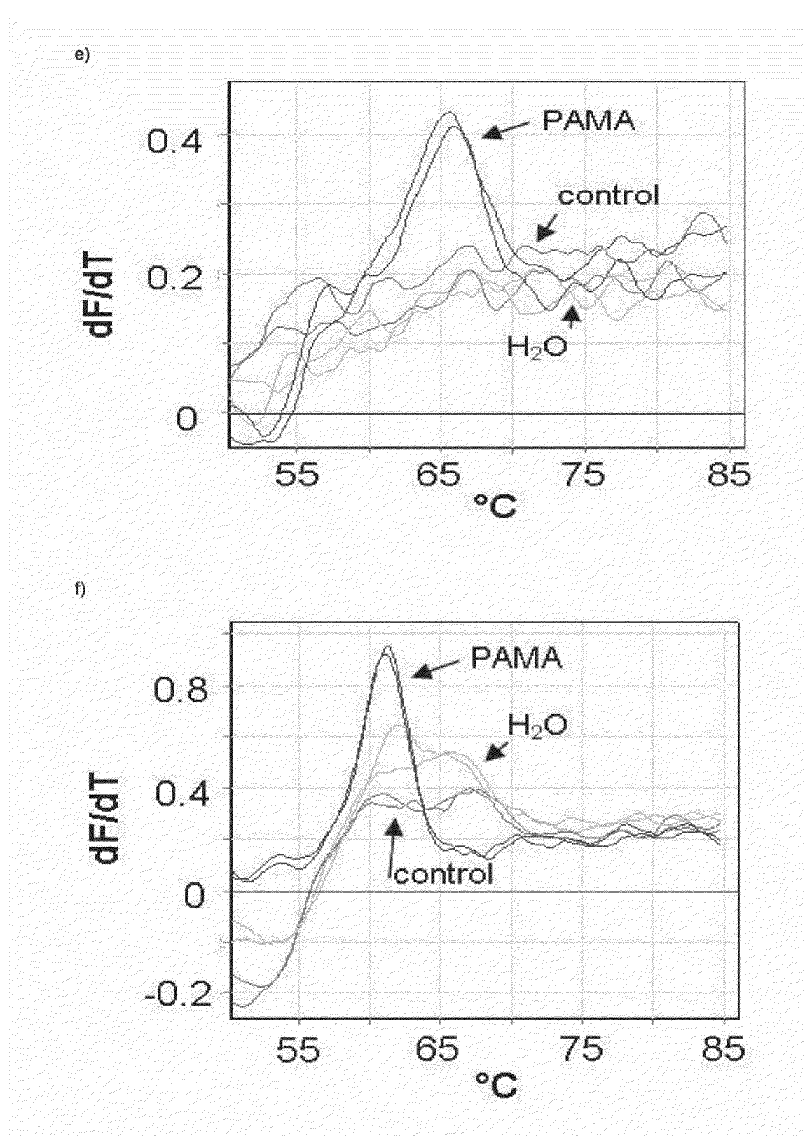

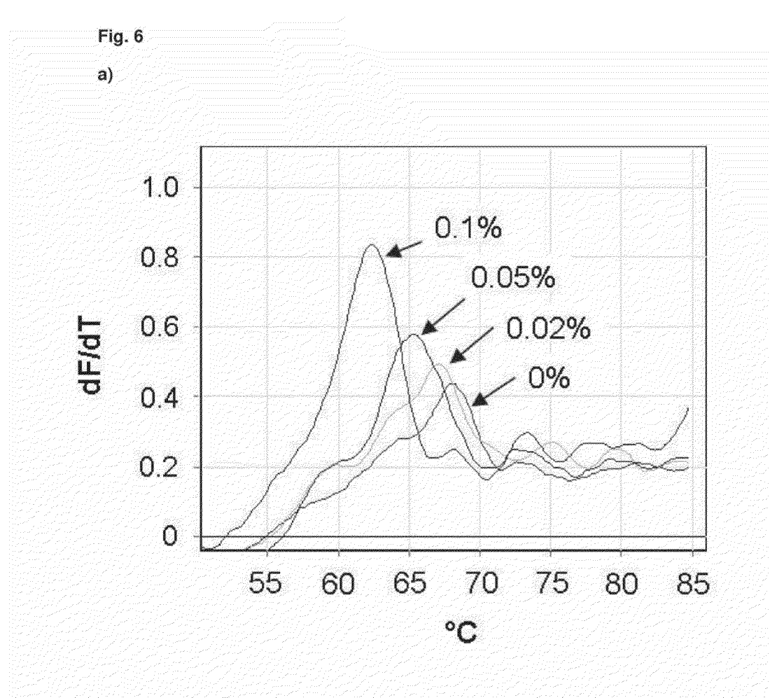

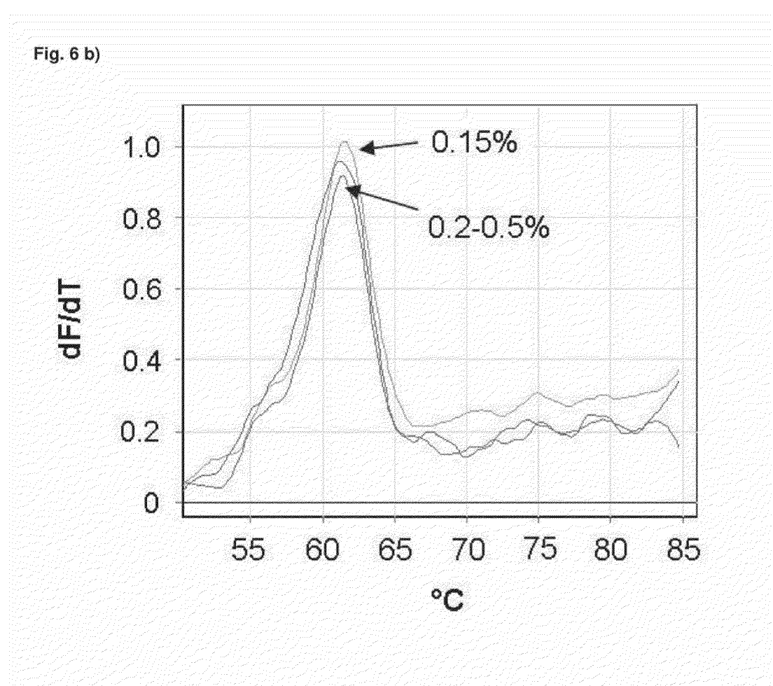

FIG. 6 shows the melting profiles obtained from analysis of symmetric PCR reactions supplemented with 0-0.1% (w/v) PAMA (see FIG. 6a) and 0.15-0.5% (w/v) PAMA (see FIG. 6b). As can be seen, already minor amounts of PAMA in the analytical samples show a significant advantage, wherein, however, better results were achieved at higher concentrations. The highest and most defined melting peak defining the optimum PAMA concentration was obtained with 0.15% (w/v) PAMA in the analytical sample.

### Example 6

### Analysis of Different Betaine and EDTA Concentrations

Furthermore, the influence of different concentrations of betaine and EDTA on the melting performance of analytical samples comprising unpurified symmetric PCR products was analysed (see example 5). To set up the analytical samples, the obtained symmetric PCR products were supplemented with 0.15% (w/v) PAMA, 100 nM probe and either 0-1000 mM betaine (0, 150, 250, 400, 525, 650, 800, 1000 mM) or 0-20 mM EDTA (0, 0.25, 0.5, 1, 2, 5, 10, 20 mM). From the amplification reaction, an additional amount of betaine (140 mM) was carried over into the analytical sample. Melting curves were recorded as described above.

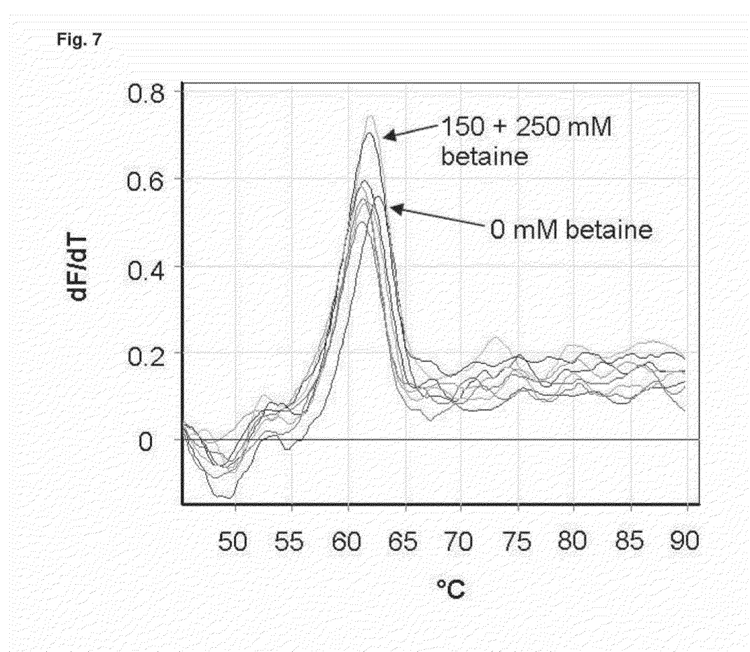

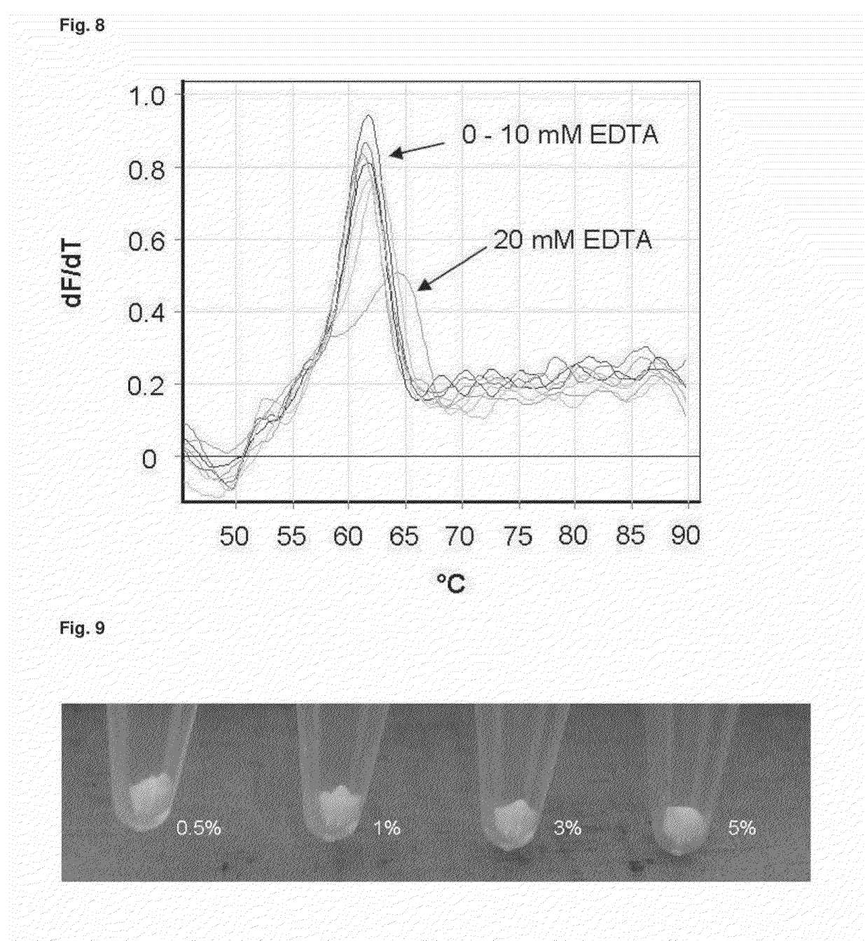

FIG. 7 shows the melting curve profiles obtained from symmetric PCR reactions supplemented with PAMA and varying amounts of betaine. As can be seen, the addition of higher concentrations of betaine improved the melting curve profile, even though under the tested conditions, only a slight improvement was seen. Thus, evidently, the significant improvement seen is attributable to the addition of PAMA. FIG. 8 shows the results for different concentrations of EDTA. No effect on melting curve performance was seen for 0-10 mM EDTA, while higher concentrations of 20 mM EDTA even had a negative effect.

In summary, the concentration optimum for PAMA was determined for melting curve analysis of symmetric PCR products using a dual labelled oligonucleotide probe. Best performance was obtained when PAMA was used in a final concentration of at least 0.1%, preferably at least 0.15% (w/v). The positive effect of PAMA on melting curve performance was even further increased when PAMA was present together with betaine in the analytical sample.

### Example 6

### Freeze-Dried PAMA/Probe Mixtures May be Supplied for Automated Workflows and “Lab-on-a-Chip” Systems

The following experiments were performed in order to provide an improved protocol for PAMA and probe delivery. In practical terms, the hybridization probe can be present in a PCR reaction already during amplification. However, it was found by the inventors that the addition of the detection probe after the PCR reaction resulted in much less background fluorescence during melting curve analysis and thus could provide better signals. Hence, better melting curve results were achieved when the probe was added after PCR amplification. Furthermore, it is preferred that PAMA is added after completion of the amplification reaction.

However, adding the detection probe and PAMA to the PCR reaction after amplification requires an additional interaction by the researcher. One possibility to simplify this additional interaction is to provide the detection probe along with PAMA in form of a single composition, such as a freeze-dried pellet or bead. Furthermore, also for “lab-on-a-chip” systems it would be an advantage if the reagents are provided in freeze-dried form. Thus, it was tested whether PAMA can be freeze-dried in different concentrations. FIG. 9 shows pellets of freeze-dried PAMA. Initial concentrations were 0.5%, 1%, 3% and 5%. All pellets of freeze-dried PAMA were quickly and entirely resolved when brought into contact with water. Hence, PAMA can be easily provided in suitable concentrations in a freeze-dried form. Furthermore, PAMA can be used as effective “bulking agent”. This means, that freeze-dried PAMA has a certain corpus that is relatively easy to handle for a researcher and that may be transferred in form of a little beadlet from one tube to another.

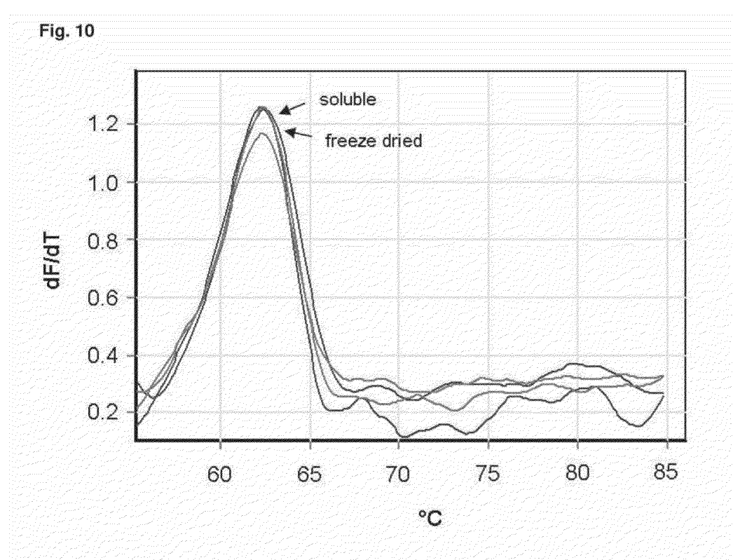

In a second experiment PAMA was freeze-dried together with a dual labelled detection probe. The freeze-dried substances were then added to a symmetric PCR product after completion of the amplification reaction to set up the analytical sample. As a control, symmetric PCR reactions were supplemented with PAMA and a dual labelled probe in solution. Melting curve analysis was performed as described above. FIG. 10 shows the melting curve profiles obtained from symmetric PCR reactions supplemented with PAMA and a dual labelled probe provided in freeze-dried form and in solution. No differences in melting behaviour could be detected. As the melting curves for both procedures assayed were largely indistinguishable, it was concluded that the freeze-drying process did not have a negative influence on the obtained quality of the data.

1: A method for performing a probe-based melting curve analysis comprising steps:
(a) preparing an analytical sample which comprises
(i) at least one double-stranded target amplicon comprising a target strand and a complementary strand,
(ii) a compound (A), which is a water-soluble polyanionic co-polymer comprising maleic acid, and
(iii) at least one detection probe or at least one detection probe set capable of hybridizing to the target strand of the target amplicon;
(b) providing in the analytical sample a double-stranded target duplex comprising the detection probe or detection probe set hybridized to the target strand; and
(c) gradually heating the analytical sample and measuring dissociation of the double-stranded target duplex during heating.

2: The method according to claim 1, wherein compound (A) is a water-soluble polyanionic co-polymer consisting of two monomeric species, wherein one of the two monomeric species is maleic acid and an other of the two monomeric species comprises at least one carboxyl group.

3: The method according to claim 1, wherein compound (A) is poly(acrylic acid-co-maleic acid).

4: The method according to claim 1, wherein preparation of the analytical sample in step (a) comprises performing an amplification reaction to produce at least one double-stranded target amplicon and adding:
aa) compound (A); and/or
bb) the at least one detection probe or the at least one detection probe set: to the produced double-stranded target amplicon.

5: The method according to claim 1, wherein the method has one or more characteristics selected from the group consisting of:
a) compound (A) is added in a form of a salt to prepare the analytical sample;
b) compound (A) has an average molecular weight in a range from 2,000 Da to 300,000 Da;
c) the analytical sample comprises compound (A) in a concentration of at least 0.02% (w/v); and
d) the analytical sample comprises compound (A) in a concentration in a range of 0.02% (w/v) to 5% (w/v).

6: The method according to claim 1, wherein the method has one or more characteristics selected from the group consisting of:
a) preparation of the analytical sample in step (a) comprises performing a symmetric amplification reaction to produce the at least one double-stranded target amplicon;
b) the target amplicon is not purified or is only partially purified after amplification and prior to performing the melting curve analysis; and
c) a multiplex melting curve analysis is performed and wherein the analytical sample comprises two or more different double-stranded target amplicons and wherein the analytical sample comprises for each target amplicon at least one detection probe or detection probe set.

7: The method according to claim 1, wherein the method is a fluorescence based melting curve analysis.

8: The method according to claim 1, wherein the detection probe or probes comprised in the detection probe set are labelled and have one or more characteristics selected from the group consisting of:
a) the label of the detection probe or probes comprised in the detection probe set is a reporter;
b) the detection probe or probes comprised in the detection probe set is/are labelled with a fluorophore and a quencher suitable to quench fluorescence of the fluorophore when the probe is not hybridized to the target strand;
c) a detection probe set is used which comprises FRET probes; and
d) the detection probe or the probes of the detection probe set span a target region of the target strand.

9: The method according to claim 1, wherein the analytical sample additionally comprises betaine.

10: A method for conducting a fluorescence based melting curve analysis, comprising steps:
(a) performing an amplification reaction to produce at least one double-stranded target amplicon, wherein the amplification reaction is a symmetric polymerase chain reaction, and adding poly(acrylic acid-co-maleic acid) and at least one detection probe or at least one detection probe set to the produced double-stranded target amplicon, thereby providing an analytical sample which comprises
(i) the at least one double-stranded target amplicon comprising a target strand and a complementary strand,
(ii) the at least one detection probe or at least one detection probe set capable of hybridizing to the target strand of the target amplicon, and
(iii) a compound (A), which is the poly(acrylic acid-co-maleic acid).
(b) providing in the analytical sample a double-stranded target duplex comprising the detection probe or detection probe set hybridized to the target strand, wherein a fluorescent signal is emitted when the double-stranded duplex is formed; and
(c) gradually heating the analytical sample and measuring dissociation of the double-stranded target duplex during heating by recording a decrease in fluorescence.

11: A method for amplifying and detecting a target nucleic acid, comprising:
amplifying a target sequence of the target nucleic acid thereby providing a double-stranded target amplicon; and
performing a melting curve analysis as defined in claim 1.

12: The method according to claim 11, wherein the method has one or more characteristics selected from the group consisting of:
a) a symmetric amplification reaction is performed;
b) the amplification reaction is a polymerase chain reaction;
c) nucleic acids are purified from a biological sample and at least an aliquot of the purified nucleic acid is used as a template for the amplification reaction; and
d) the method is for detecting a presence or an absence of a pathogen in a sample, to detect and/or categorize genetic mutations, SNP genotyping, tumor typing, to identify new genetic variants without sequencing, to determine genetic variation in a population prior to sequencing, mutation discovery, heterozygosity screening, DNA finger printing, haplotype blocks characterization, DNA methylation analysis, DNA mapping, species identification, pathogen detection, viral/bacterial population diversity investigation and HLA compatibility typing, high resolution single nucleotide polymorphism (SNP) mapping, genotyping of diseases, forensic analysis, disease diagnostics, individual identification and/or mutational screening.

13: A composition comprising at least one detection probe or at least one detection probe set and a compound (A) which is a water-soluble polyanionic co-polymer comprising maleic acid.

14: The composition of claim 13, wherein the composition has one or more characteristics selected from the group consisting of:
a) compound (A) is a water-soluble polyanionic co-polymer consisting of two monomeric species, wherein one of the two monomeric species is maleic acid and an other of the two monomeric species comprises a carboxyl group;
b) compound (A) is poly(acrylic acid-co-maleic acid);
c) the detection probe or detection probe set has are labelled, and the label of the detection probe or probes comprised in the detection probe set is a reporter;
d) the composition is a dried composition;
f) the composition comprises betaine;
g) the composition comprises at least one double-stranded target amplicon wherein the target amplicon was obtained in a symmetric amplification reaction;
h) the composition comprises reagents from an amplification reaction selected from a polymerase, primers, dNTPs, and ions;
i) the detection probe or probes comprised in the detection probe set is/are labelled with a fluorophore and a quencher suitable to quench fluorescence of the fluorophore when the probe is not hybridized to the target strand;
j) a detection probe set is used which comprises FRET probes; and
k) the detection probe or the probes of the detection probe set span a target region of the target strand.

15: A kit for performing a melting curve analysis, comprising at least one detection probe or detection probe set and a compound (A), which is a water-soluble polyanionic co-polymer comprising maleic acid.

16: The kit according to claim 15, wherein the kit has one or more characteristics selected from the group consisting of:
a) compound (A) is a water-soluble polyanionic co-polymer consisting of two monomeric species, wherein one of the two monomeric species is maleic acid and an other of the two monomeric species comprises a carboxyl group;
b) compound (A) is poly(acrylic acid-co-maleic acid); and
c) compound (A) and the at least one detection probe or at least one detection probe set are comprised in a same composition.

17. (canceled)

18: The method according to claim 1, wherein the analytical sample further comprises the target strand as a single-stranded amplicon.

19: The method according to claim 7, wherein fluorescence decreases when the detection probe or detection probe set dissociates from the target strand.

20: The method according to claim 10, wherein the analytical sample further comprises the target strand as a single-stranded amplicon.
